(19) United States
(12) Patent Application Publication
(10) Pub. No.: US 2026/0355009 A1
(43) Pub. Date: Nov. 12, 2026

(54) TEMPERATURE CONTROL SYSTEM FOR A THERMAL ACCESSORY THAT USES A GLYCOL-BASED FLUID

(71) Applicant: CardioQuip, LLC, College Station, TX (US)

(72) Inventors: William STEWART, Golden, CO (US); Anna STEWART, Golden, CO (US); John DEAN, Austin, TX (US); Kayla WILLIS, Bryan, TX (US); Daniel CORONEL, Maple Grove, MN (US); Valerie COTE, Santa Clara, CA (US)

(21) Appl. No.: 19/747,757

(22) Filed: Jul. 20, 2026

Related U.S. Application Data
(63) Continuation-in-part of application No. 19/648,238, filed on Apr. 15, 2026.
Application No. 19/648,238 is a continuation-in-part of application No. 17/954,273, filed on Sep. 27, 2022, now abandoned.
(60) Provisional application No. 63/257,001, filed on Oct. 18, 2021.

(51) Int. Cl. A61F 7/00

(52) U.S. Cl. CPC A61F 7/0097; A61F 2007/0054

(57) ABSTRACT

Disclosed herein is a technology for controlling a temperature of a thermal accessory with a circulating glycol-based thermal liquid. The thermal liquid comprises at least a mixture of water and a monomeric glycol, providing antimicrobial properties that inhibit microbial growth within the fluid path. A fluid path of the technology includes a liquid pump, a liquid reservoir, a heating component, and a cooling component. A first portion of the fluid path includes a valve configured to restrict or permit the thermal liquid from entering the liquid reservoir based on whether the thermal accessory is coupled to the system. A second portion of the fluid path includes the output port. A control unit manages the temperature of the thermal accessory through controlled activation of the heating component or the cooling component.

### CROSS-REFERENCE TO RELATED APPLICATIONS

This application is a continuation in part of US Application No. 19/648,238, titled “System and Method for Heating and Cooling a Thermal Accessory” and filed on Apr. 15, 2026, which is a continuation in part of US Application No. 17/954,273, titled “Thermal Accessory Temperature Controller Using Glycol” and filed on Sep. 27, 2022, which claims priority to US Provisional Application No. 63/257,001, titled “Thermal Blanket System Using Propylene Glycol” and filed on Oct. 18, 2021, each of which is incorporated by reference in its entirety.

### BACKGROUND

The clinical importance of patient temperature control has been recognized for decades. Early temperature management techniques were relatively rudimentary and included passive warming measures such as blankets, reflective covers, or warmed ambient air within operating rooms. As surgical procedures increased in duration and complexity, and as clinical understanding of perioperative hypothermia and hyperthermia improved, more active systems were developed. These early active systems typically relied on forced-air warming devices or circulating water blankets that transferred heat through conduction or convection at the patient’s skin surface. Such systems marked a transition from passive thermal support to controlled thermal therapy.

Subsequent generations of patient temperature management systems incorporated improved fluid circulation assemblies, more precise heating and cooling elements, and electronic controllers capable of maintaining a target setpoint. In some configurations, temperature-controlled liquids were circulated through pads, wraps, or garments placed in contact with the patient, while other systems employed temperature-conditioned air delivered through specialized blankets. In critical care and cardiac applications, blood-contacting heat exchange devices were also introduced to provide more direct modulation of core body temperature. These developments reflected an effort to achieve faster response times, improved temperature stability, and enhanced patient safety.

Despite these incremental improvements, much of the foundational architecture of patient temperature management systems has remained largely unchanged for many years. Many commercially available systems continue to rely on heavy, cumbersome components, resulting in larger devices. Further, current systems utilize control strategies based primarily on relatively simple feedback loops responsive to one or more temperature sensors. Further improvements in performance have often been limited by constraints associated with thermal inertia, heat transfer efficiency at the skin interface, user workflow considerations, regulatory requirements, and manufacturing cost. Consequently, innovation in the field has tended to focus on refinements of existing platforms rather than fundamental redesigns of system architecture or control methodologies.

Moreover, clinical and regulatory considerations have influenced the pace and direction of development. Because temperature management devices directly affect core physiological parameters, they are subject to stringent safety standards and validation requirements. The need to ensure reliability, avoid thermal injury, and maintain compatibility with diverse clinical environments has encouraged conservative design evolution. As a result, although temperature management remains a critical component of perioperative care, therapeutic hypothermia, and intensive care treatment, there remains a need for systems that provide enhanced responsiveness, improved efficiency, and more adaptable control strategies.

### BRIEF DESCRIPTION OF THE DRAWINGS

Several implementations of the present invention will be described and explained through the use of the accompanying drawings.

FIG. 1 is a block diagram illustrating the components comprising a patient temperature management system according to various aspects of the present technology.

FIG. 2 illustrates a patient temperature management system in a representative application according to various aspects of the present technology.

FIG. 3 illustrates in greater detail the thermal blanket of FIG. 2 according to various aspects of the present technology.

FIG. 4A is a block diagram illustrating a fluid path of a thermal accessory controller according to various aspects of the present technology.

FIG. 4B is a block diagram illustrating data channels of a thermal accessory controller according to various aspects of the present technology.

FIG. 5 is a flow diagram illustrating the operation of the microcontroller unit (MCU) according to one embodiment of the disclosure.

FIG. 6 is a flow diagram illustrating the operation of the proportional-integral-derivative (PID) algorithm and other algorithms performed by the microcontroller unit (MCU) in the thermal blanket controller according to one embodiment of the disclosure.

FIG. 7 illustrates a conduit used to decontaminate the thermal blanket controller according to one embodiment of the disclosure.

FIG. 8 is a flow diagram illustrating the operation of the decontamination procedure of the thermal accessory controller according to one embodiment of the disclosure.

FIG. 9 is a flowchart of a process for priming a patient temperature management system according to various aspects of the present technology.

FIG. 10 is a flowchart of a process for pre-warming or pre-cooling fluid in a patient temperature management system according to various aspects of the present technology.

FIG. 11 is a flowchart of a process for monitoring a temperature of a fluid in a patient temperature management system when a thermal accessory is attached according to various aspects of the present technology.

FIG. 12 is a block diagram that illustrates an example of a computer system in which at least some operations described herein can be implemented

The technologies described herein will become more apparent to those skilled in the art from studying the Detailed Description in conjunction with the drawings. Embodiments or implementations describing aspects of the present technology are illustrated by way of example and not limitation. While the drawings depict various implementations for the purpose of illustration, those skilled in the art will recognize that alternative implementations can be employed without departing from the principles of the disclosure. Accordingly, while specific implementations are shown in the drawings, the technology is amenable to various modifications.

### DETAILED DESCRIPTION

Patient temperature management devices (or simply “devices”) are medical systems designed to regulate the body temperature of patients in various clinical settings. These devices operate by controlling the temperature of a fluid, such as air or liquid, that is circulated through one or more accessories that contact or interface with a patient's body. Temperature management may involve warming a patient, cooling a patient, or maintaining a patient at a target temperature, depending on the clinical application. Such devices find use across a range of medical contexts where precise control of patient temperature is therapeutically necessary or beneficial.

Patient temperature management devices generally include recirculating fluid flows - for example, air currents or liquid paths - that control the temperature of a thermal accessory, such as a heat exchanger, extracorporeal oxygenator, thermal blanket, or thermal wrap. These accessories may be used alone or in conjunction with other perfusion apparatuses (e.g., heart-lung machines) to control the temperature of a patient or the temperature of liquids administered to a patient. For example, a heart-lung machine circulates blood through one channel of a heat exchanger while a patient temperature management device circulates temperature-controlled liquid through a separate channel of the heat exchanger, enabling energy transfer between the liquids in the two channels without direct contact. In another example, a patient temperature management device circulates temperature-controlled liquid through a thermal blanket resting on a patient to modulate the temperature of the patient supracutaneously.

Conventional liquid-circulating patient temperature management systems utilize various heating and cooling elements in conjunction with complex software systems to stabilize the temperature of the circulating liquid. Due to differences in thermal transfer, liquid-circulating temperature management devices generally aim to maintain temperature equilibrium within ±0.5°C of the target temperature, while air-circulating devices typically exhibit less control of temperature. The enhanced stability of liquid-based devices is required for new clinical applications of long-term patient temperature management devices such as extracorporeal membrane oxygenation (ECMO) and targeted temperature management (TTM).

The selection of an appropriate thermal liquid for circulation through patient temperature management systems represents a significant design consideration that affects system performance, safety, and operational characteristics. An ideal thermal liquid exhibits high specific heat capacity to maximize thermal energy transfer per unit volume, low viscosity to minimize pumping losses and enable efficient circulation, chemical stability to prevent degradation over extended use, and compatibility with system materials to avoid corrosion or degradation of internal components. Cost considerations also favor readily available liquids that do not require specialized handling or disposal procedures, particularly where the design of the patient temperature management system contemplates periodic replacement or replenishment of the liquid over the operational life of the system .

Water has traditionally been employed as the thermal liquid in many patient temperature management systems due to its favorable thermodynamic properties. Water possesses a high specific heat capacity (~4.18 kJ/kgK) and is inexpensive, readily available, and chemically compatible with most materials used in medical device construction.

However, the use of water as a thermal liquid introduces several operational challenges that can compromise system reliability and patient safety. Water provides a favorable environment for microbial growth, particularly bacteria and fungi, which can proliferate within the fluid pathways of the system and create contamination risks. The warm, moist conditions within patient temperature management systems are conducive to the formation of biofilms on internal surfaces, including tubing walls, reservoir interiors, and heat exchanger passages. These biofilms can harbor pathogenic microorganisms that are resistant to routine cleaning procedures. In healthcare settings where patients may be immunocompromised or recovering from surgical procedures, the presence of microbial contamination in temperature management equipment poses a significant infection control concern. Consequently, water-based systems typically require frequent cleaning and disinfection cycles, the use of antimicrobial additives, or periodic replacement of fluid to maintain acceptable levels of microbial contamination, all of which increase operational burden, associated costs, and risk of noncompliance with maintenance.

Additionally, water has a freezing point of 0°C, which limits the minimum achievable temperature for patient cooling applications and creates a risk of ice formation within the system if temperatures approach or fall below freezing. Ice formation within fluid pathways can obstruct flow, damage pumps and other components, and compromise system operation during critical patient cooling procedures. Water has a boiling point of 100°C at atmospheric pressure, which constrains the maximum temperature achievable during high-temperature disinfection procedures. This boiling point limitation presents challenges for thermal disinfection protocols, as temperatures approaching the boiling point can cause vaporization, loss of solution volume, and the formation of steam pockets that may damage system components or create inconsistent thermal transfer characteristics.

To address these limitations, the present technology employs a monomeric, glycol-based mixture with water as the thermal liquid. Glycols are synthetic organic compounds that are generally recognized as safe for use in medical, food, and automotive applications. When combined with water, glycols create a solution that retains favorable thermal properties while mitigating the disadvantages associated with pure water.

For example, the glycol-water mixture exhibits antimicrobial properties that inhibit the growth of bacteria and other microorganisms within the fluid pathways. These antimicrobial properties arise from several mechanisms, including the osmotic stress imposed on microbial cells by the glycol concentration, the reduction in water activity within the solution, and the disruption of microbial cell membrane integrity caused by the chemical structure of glycols. The presence of glycols creates an environment that is less hospitable to microbial proliferation compared to pure water, thereby reducing contamination risks and extending the intervals between required cleaning and disinfection procedures.

As another example, the addition of propylene glycol depresses the freezing point of the solution, enabling the system to achieve lower temperatures for patient cooling applications without risk of ice formation. Conversely, the glycol-water mixture has an elevated boiling point compared to pure water, permitting the system to circulate thermal liquid at temperatures exceeding 70°C during high-temperature disinfection procedures with reduced risk of vaporization or loss of solution volume. For example, propylene glycol has a vapor pressure of approximately 0.13 mmHg at 25 °C, while water's vapor pressure is 23.8 mmHg at 25 °C. At elevated temperatures, such as 70 °C, vapor pressure increases exponentially, meaning that water will be more susceptible to losses and evaporation, while propylene glycol will be more likely to remain.

The adoption of a glycol-based thermal liquid, though, fundamentally alters the thermal management characteristics of the patient temperature management device. The specific heat capacity and thermal conductivity of the glycol-water mixture differ from those of pure water, requiring adjustments to control algorithms and heating and cooling element sizing to achieve equivalent thermal performance. The viscosity of the glycol-water mixture is higher than that of pure water, which affects pump selection and flow rate calculations. Furthermore, the antimicrobial properties of the glycol-water mixture enable devices requiring no disinfection of the internal fluid path..

The present technology addresses these considerations through an integrated system architecture that accounts for the altered thermophysical properties of the glycol-water mixture. The control algorithms implemented within the system are configured to compensate for the reduced specific heat capacity and thermal conductivity of the glycol-water solution relative to pure water, ensuring that target temperatures are achieved within clinically acceptable timeframes despite the different thermal response characteristics. The liquid pump and associated fluid pathways are sized to accommodate the increased viscosity of the glycol-water mixture while maintaining adequate flow rates for effective thermal transfer at the patient interface. Additionally, the system incorporates a high-temperature disinfection mode that circulates the glycol-water mixture at temperatures of approximately 70°C or higher, leveraging the elevated boiling point of the solution to achieve effective microbial decontamination without the vaporization concerns that would preclude such procedures in water-based systems. This combination of glycol-based thermal liquid and adapted control strategies is expected to provide a patient temperature management system with reduced microbial contamination risk, extended operational temperature range, and simplified maintenance requirements compared to conventional water-based systems.

The description and associated drawings are illustrative examples and are not to be construed as limiting. This disclosure provides certain details for a thorough understanding and enabling description of these examples. One skilled in the relevant technology will understand, however, that the invention can be practiced without many of these details. Likewise, one skilled in the relevant technology will understand that the invention can include well-known structures or features that are not shown or described in detail, to avoid unnecessarily obscuring the descriptions of examples.

### Systems and Methods for Heating and Cooling a Thermal Accessory

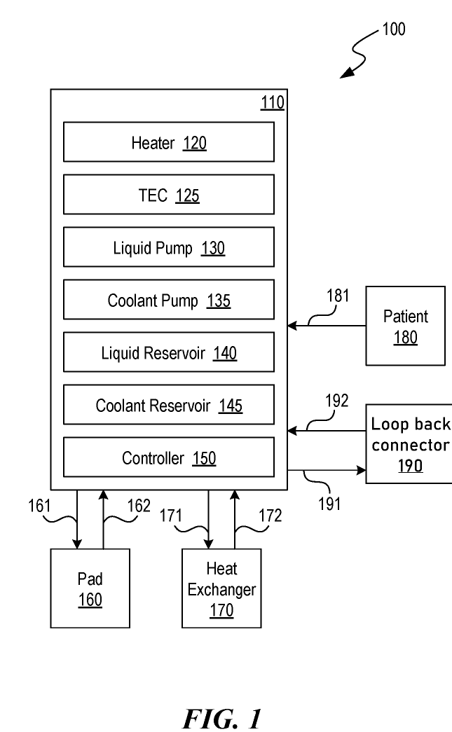

FIG. 1 is a block diagram illustrating the components comprising a patient temperature management system 100. Note that FIG. 1 depicts features that are described as functional units. This grouping has been done to discern between operations of the system and should not be taken as an implication that the units must be distinct or separate hardware components.

The patient temperature management system 100 comprises a thermal accessory controller (TAC) 110, which includes a heater 120, a TEC block 125, liquid pump 130, coolant pump 135, liquid reservoir 140, coolant reservoir 145, and a controller 150. The heater 120 is a heating element configured to increase the temperature of the thermal liquid circulating through the system. In various embodiments, the heater 120 comprises a resistive heating element, an immersion heater, a stainless steel heater chamber, a cartridge heater, or other suitable heating device capable of transferring thermal energy to the circulating liquid. The TEC block 125 is a solid-state cooling device that utilizes the Peltier effect to transfer heat from one side of the device to the other when an electrical current is flowing. In various embodiments, the TEC block 125 comprises one or more Peltier modules, thermoelectric heat pumps, or semiconductor-based cooling elements arranged in series or parallel configurations.

The liquid pump 130 is a fluid displacement device configured to circulate the thermal liquid through a fluid path of the TAC 110 and connected accessories. In various embodiments, the liquid pump 130 may comprise a centrifugal pump, a peristaltic pump, a diaphragm pump, a gear pump, or other suitable pumping mechanism capable of providing consistent fluid flow. The coolant pump 135 is a fluid displacement device configured to circulate coolant liquid through a heat dissipation circuit that removes waste heat from the TEC block 125. In various embodiments, the coolant pump 135 comprises a centrifugal pump, a magnetic drive pump, an impeller pump, or other suitable pumping mechanism.

The liquid reservoir 140 is a storage vessel configured to hold a volume of thermal liquid that circulates through the TAC 110 and connected accessories. In various embodiments, the liquid reservoir 140 comprises a tank, a bladder, a chamber, or other suitable container capable of storing the thermal liquid and providing thermal mass to stabilize temperature fluctuations. The coolant reservoir 145 is a storage vessel configured to hold a volume of coolant liquid that circulates through the heat dissipation circuit. In various embodiments, the coolant reservoir 145 comprises a tank, a bladder, a chamber, or other suitable container.

Finally, the controller 150 is a processing unit configured to manage the overall operation of the TAC 110, including regulating power to the heater 120 and TEC block 125, controlling the liquid pump 130 and coolant pump 135, and processing temperature data from connected sensors. In various embodiments, the controller 150 comprises a microcontroller unit (MCU), a microprocessor, a programmable logic controller (PLC), an application-specific integrated circuit (ASIC), a field-programmable gate array (FPGA), or other suitable processing device.

The patient temperature management system 100 also includes attachable accessories such as a thermal accessory 160, a heat exchanger 170, and a loop back connector 190. The thermal accessory 160 is a patient-contacting device configured to transfer thermal energy between the circulating thermal liquid and a patient. In various embodiments, the thermal accessory 160 comprises a thermal blanket, a thermal pad, a thermal wrap, a thermal garment, a cooling cap, a body suit, or other suitable device that interfaces with a patient's body to provide heating or cooling. Though illustrated in FIG. 1 as a single thermal accessory 160, the present technology is not so limited. Accordingly, the TAC 110 may interface with two or more thermal accessories 160 as well (e.g., 2, 3, or more thermal accessories each applied to one or more patients). The heat exchanger 170 is a device configured to transfer thermal energy between the circulating thermal liquid and a separate fluid circuit without direct mixing of the fluids. In various embodiments, the heat exchanger 170 comprises a shell-and-tube heat exchanger, a plate heat exchanger, a coaxial heat exchanger, an extracorporeal oxygenator, or other suitable device used in conjunction with perfusion apparatuses such as heart-lung machines or ECMO circuits.

The loop back connector 190 is a conduit configured to provide a direct liquid path between the output and input of the TAC 110 when a thermal accessory is not connected, which – in some embodiments – provides fluid circulation during priming, cleaning, or disinfection procedures. In various embodiments, the loop back connector 190 comprises a flexible hose, a rigid tube, a curved conduit, or other suitable connector that couples the output and input ports of the TAC 110.

The thermal accessory 160, the heat exchanger 170, and loop back connector 190 are connected to the TAC 110 by means of input liquid conduits (e.g., hoses) 161, 171, and 191, respectively, and output liquid conduits 162, 172, and 192, respectively. The input conduit 161 is a fluid pathway configured to carry temperature-controlled thermal liquid from the TAC 110 to the thermal accessory 160. The output conduit 162 is a fluid pathway configured to return thermal liquid from the thermal accessory 160 back to the TAC 110. Similarly, the input conduit 171 and output conduit 172 provide fluid pathways between the TAC 110 and the heat exchanger 170, while the input liquid conduit 191 and output liquid conduit 192 provide fluid pathways between the TAC 110 and the loop back connector 190. In various embodiments, the conduits 161, 162, 171, 172, 191, and 192 comprise flexible tubing, reinforced hoses, insulated conduits, or other suitable fluid pathways.

In some embodiments, temperature data 181 from a patient 180 is input into the TAC 110. The temperature data 181 represents measurements of the patient's body temperature obtained from one or more temperature sensors. In various embodiments, the temperature data 181 is obtained from an oral thermometer, a rectal thermometer, an esophageal thermometer, a skin thermometer, a tympanic thermometer, a bladder temperature probe, or other suitable temperature sensing device.

The thermal liquid circulating through the TAC 110 and connected accessories can be a glycol-based mixture. In various embodiments, the glycol-based mixture comprises propylene glycol and water in a predetermined ratio. The concentration of propylene glycol in the mixture may range from approximately 20% to approximately 100% by volume, with the specific concentration selected based on the desired thermophysical properties and operational temperature range. In some embodiments, the glycol-based mixture comprises approximately 30% to approximately 70% propylene glycol by volume, which provides a balance between antimicrobial effectiveness, viscosity, and thermal transfer efficiency. For example, the glycol-based mixture comprises approximately 60-70% propylene glycol by volume when an application of the TAC 110 requires greater antimicrobial effect (e.g., in an environment where the thermal liquid of the TAC 110 will be replaced infrequently). As another example, the glycol-based mixture comprises approximately 50% propylene glycol by volume when an application of the TAC 110 requires a balance of the antimicrobial effect with retention of the thermal- and viscosity-related characteristics of water. As a further example, the glycol-based mixture comprises approximately 30-40% propylene glycol by volume when an application of the TAC 100 requires greater retention of the thermal- and viscosity-related characteristics of water (e.g., in an environment where the thermal liquid of the TAC 110 will be replaced frequently and the thermal transfer efficiency of water is advantageous).

The use of a glycol-based thermal liquid provides several advantages over pure water systems. First, the glycol-based mixture exhibits inherent antimicrobial properties that inhibit the growth of bacteria, fungi, and other microorganisms within the fluid pathways of the TAC 110. These antimicrobial properties arise from multiple mechanisms, including the osmotic stress imposed on microbial cells by the glycol concentration, the reduction in water activity within the solution, and the disruption of microbial cell membrane integrity caused by the chemical structure of glycols. The presence of glycols creates an environment that is less hospitable to microbial proliferation compared to pure water, thereby reducing contamination risks and extending the intervals between required cleaning and disinfection procedures or removing cleaning and disinfection altogether.

Second, the addition of glycols depress the freezing point of the thermal liquid, enabling the TAC 110 to achieve lower temperatures for patient cooling applications without risk of ice formation within the fluid pathways. Depending on the concentration of glycol, the freezing point of the glycol-based mixture may be depressed to temperatures below -20°C, providing a substantial margin of safety against ice formation during aggressive cooling protocols. This freezing point depression eliminates the risk of ice-induced flow obstructions, pump damage, and component failures that can occur in water-based systems operating at low temperatures.

The thermophysical properties of the glycol-based mixture differ from those of pure water in ways that affect system design and control strategies. The specific heat capacity of the glycol-based mixture is lower than that of pure water, meaning that less thermal energy is required to change the temperature of a given volume of the mixture. For example, a 50% propylene glycol solution has a specific heat capacity of approximately 3.4 kJ/kgK compared to approximately 4.18 kJ/kgK for pure water. This reduced specific heat capacity affects the thermal response characteristics of the TAC 110 and requires adjustments to control algorithms to achieve equivalent temperature regulation performance.

The thermal conductivity of the glycol-based mixture is also lower than that of pure water, which affects the rate of heat transfer between the thermal liquid and the heating and cooling elements within the TAC 110, as well as the rate of heat transfer at the patient interface through the thermal accessory 160. For example, a 50% propylene glycol solution has a thermal conductivity of approximately 0.36 W/mK compared to approximately 0.60 W/mK for pure water. This reduced thermal conductivity means that heat transfers more slowly between the thermal liquid and adjacent surfaces, which can affect the responsiveness of the system to changes in heating or cooling demand. However, the reduced thermal conductivity, in combination with the reduced specific heat capacity, also means that the glycol-based mixture can be heated or cooled more rapidly than pure water for a given heat input or removal rate, as less thermal energy must be transferred to achieve a given temperature change. The controller 150 compensates for these altered thermal properties through modified control algorithms that account for the different thermal response characteristics of the glycol-based mixture.

The viscosity of the glycol-based mixture is higher than that of pure water, particularly at lower temperatures. This increased viscosity affects pump selection, flow rate calculations, and pressure drop characteristics throughout the fluid pathways. The liquid pump 130 is sized to accommodate the increased viscosity of the glycol-based mixture while maintaining adequate flow rates for effective thermal transfer at the patient interface. In various embodiments, the liquid pump 130 provides sufficient pumping capacity to overcome the increased flow resistance caused by the higher viscosity of the glycol-based mixture across the entire operational temperature range of the TAC 110.

In some embodiments, the water and glycol mixture of the thermal liquid also includes one or more additives. For example, propylene glycol solutions tend to exhibit mildly acidic characteristics, with pH values that can decrease over time as the propylene glycol undergoes oxidative degradation, producing organic acids such as lactic acid, acetic acid, and formic acid that further lower the pH of the solution. The acidic environment created by degraded propylene glycol accelerates corrosion of metallic components within the fluid pathways of the TAC 110. Corrosion of these metallic components can release metal ions into the circulating thermal liquid, compromise the structural integrity of fluid pathway components, create particulate contamination that can obstruct flow passages or damage pump mechanisms, and reduce the thermal transfer efficiency of heat exchange surfaces due to the formation of corrosion products and scale deposits.

To address these corrosion concerns, the glycol-based mixture may include a corrosion inhibitor to protect metallic components within the fluid pathways of the TAC 110 from degradation over time. In various embodiments, the corrosion inhibitor maintains the glycol-based thermal liquid at a more neutral or alkaline pH. The corrosion inhibitor neutralizes acidic degradation products as they form, preventing the accumulation of organic acids that would otherwise lower the pH and accelerate corrosion. By maintaining the thermal liquid in a neutral or mildly alkaline state, the corrosion inhibitor creates an environment that is substantially less corrosive to metallic components compared to unbuffered glycol solutions. In various embodiments, the corrosion inhibitor comprises a pH buffer, phosphate-based inhibitors, silicate-based inhibitors, borate-based buffers, organic acid technology (OAT) inhibitors, amine-based pH stabilizers, or other suitable corrosion-inhibiting compounds that are compatible with the glycol-based thermal liquid.

In other embodiments, the glycol-based thermal liquid includes a pH-activated indicator configured to provide a visual indication of fluid condition. The pH-activated indicator may comprise a dye or other colorimetric compound that changes color in response to changes in the pH of the thermal liquid. Changes in pH can correlate with fluid degradation, accumulation of acidic degradation products, depletion of corrosion inhibitors, or reduced antimicrobial effectiveness of the glycol-based mixture. The addition of a pH-activated indicator allows users to easily determine when the thermal liquid is no longer suitable for use and requires replacement, without relying solely on predefined maintenance intervals or elapsed time since the last fluid change. In various embodiments, the pH-activated indicator comprises phenolphthalein, bromothymol blue, phenol red, methyl orange, universal indicator dye, or other suitable pH-sensitive compounds that are compatible with the glycol-based thermal liquid and provide a readily discernible color change within the pH range of interest.

In still further embodiments, the condition of the glycol-based thermal liquid is assessed using a pH testing strip. A user may obtain a sample of the thermal liquid from the TAC 110, such as through a sampling port or drain valve, and apply the sample to a pH testing strip to determine the pH level of the thermal liquid. Alternatively, the user may dip the pH testing strip directly into an accessible portion of the thermal liquid, such as within the liquid reservoir 140 or at an open port during maintenance procedures. The pH testing strip provides a colorimetric indication of the pH level that can be compared against a reference scale to determine whether the thermal liquid remains within acceptable pH limits. If the measured pH falls outside the acceptable range, the user can replace the thermal liquid to restore proper fluid condition and maintain the antimicrobial effectiveness and corrosion protection characteristics of the glycol-based mixture.

In some embodiments, the glycol-based thermal liquid includes additives beyond corrosion inhibitors to improve clinical performance. Such additives may comprise bactericidal chemicals that provide more rapid disinfection of the fluid pathways compared to the inherent antimicrobial properties of glycol alone. In various embodiments, the bactericidal additives comprise glutaraldehyde, alcohols such as isopropyl alcohol or ethanol, hydrogen peroxide, antimicrobial nanomaterials such as silver nanoparticles or copper nanoparticles, quaternary ammonium compounds, or other suitable disinfecting agents that are compatible with the glycol-based thermal liquid and the materials of construction within the TAC 110. The inclusion of bactericidal additives can reduce the time required for effective microbial decontamination during cleaning and disinfection procedures, enhance the antimicrobial effectiveness of the thermal liquid during normal operation, and provide an additional margin of safety against microbial contamination in healthcare settings where infection control is critical.

In other embodiments, the glycol-based thermal liquid includes additives to improve thermal performance. As described above, propylene glycol has a lower specific heat capacity than water, which can result in reduced cooling performance when compared with water-based systems for a given flow rate and temperature differential. To address this limitation, the glycol-based mixture may include other fluids or materials that increase the overall heat capacity of the thermal liquid. In various embodiments, the thermal performance additives comprise other glycols such as ethylene glycol, specialized nanomaterials such as carbon nanotubes or metal oxide nanoparticles that enhance thermal conductivity and heat capacity, phase change materials that absorb or release latent heat during temperature transitions, or other suitable fluids and materials that improve the thermal transfer characteristics of the glycol-based mixture.

In still further embodiments, the glycol-based thermal liquid includes additives to improve the useful life of the thermal liquid. Such additives may comprise stabilizers that inhibit oxidative degradation of the propylene glycol, antioxidants that scavenge free radicals and prevent chain reactions that lead to fluid breakdown, or other compounds that maintain the chemical integrity of the thermal liquid over extended periods of use. The inclusion of stabilizers can extend the intervals between required fluid replacement, reduce operational costs associated with thermal liquid maintenance, and ensure consistent thermal and antimicrobial performance throughout the service life of the thermal liquid. In yet further embodiments, the glycol-based thermal liquid includes other additives such as heat-capacity additives, corrosion inhibitors, and pH indicators.

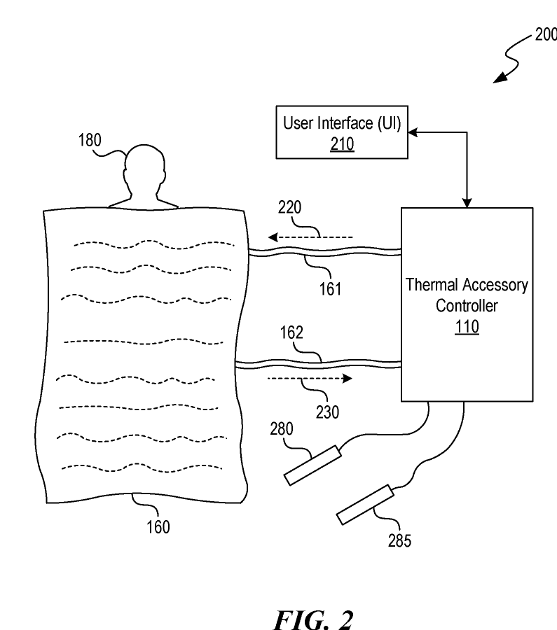

FIG. 2 illustrates a patient temperature management system 200 (e.g., the patient temperature management system 100 of FIG. 1) in a representative application. The patient temperature management system 200 comprises the TAC 110, a user interface (UI) 210, the thermal accessory 160, the input conduit 161, the output conduit 162, and temperature probes 280 and 285. The UI 210 is coupled to the TAC 110 and allows a user or operator to control the operation of the TAC 110. Across various embodiments, the UI 210 is a laptop computer, a mobile phone, a tablet device, a human-machine interface integrated into or in communication with TAC 110, or another medical device that communicates by wireline or wirelessly with the TAC 110.

The temperature probe 280 and temperature probe 285 are coupled to the TAC 110 and are configured to measure the temperature of the patient 180. Across various embodiments, the temperature probes 280 and 285 are oral thermometers, rectal thermometer, esophageal thermometers, skin thermometers, tympanic thermometers, bladder temperature probes, or other suitable temperature sensing devices. The TAC 110 reads temperature recordings from temperature probes 280 and 285 and, in response, may increase (heat) or decrease (cool) the temperature of a thermal liquid that circulates through the thermal accessory 160. In a heating mode, the warmed thermal liquid provides thermal energy to the patient 180 through the thermal accessory 160 (e.g., a thermal blanket warming the patient 180 supracutaneously). In a cooling mode, the cooled thermal liquid absorbs thermal energy from the patient 180 through the thermal accessory 160.

As indicated by the directional liquid-flow arrow 220, the input conduit 161 (e.g., a hose 161) carries temperature-controlled thermal liquid from the TAC 110 to the thermal accessory 160. As indicated by the directional liquid-flow arrow 230, the output conduit 162 (e.g., a hose 162) returns the temperature-controlled thermal liquid from the thermal accessory 160 back to the TAC 110. The returned thermal liquid may then be rewarmed or cooled by the TAC 110 as needed.

In the example embodiment of FIG. 2, the thermal accessory 160 comprises a blanket that covers the body of the patient 180. However, this is by way of example only and should not be construed to limit the scope of the disclosure or the claims below. In alternate embodiments, the thermal accessory 160 comprises a pad on which the patient 180 lies, a garment that the patient 180 wears, or a heat exchanger. For the sake of clarity and conciseness, the following descriptions shall assume that the thermal accessory 160 comprises a thermal blanket 160.

In some embodiments, the TAC 110 uses an antimicrobial liquid (e.g., a glycol-based thermal liquid as described above with respect to FIG. 1) to prevent microbes (e.g., bacteria) from growing and proliferating in the liquid path of the TAC 110. In an example embodiment of the present disclosure, the thermal liquid comprises a propylene glycol and water solution. The propylene glycol concentration may be selected to provide a desired balance of antimicrobial effectiveness, freezing point depression, boiling point elevation, and thermal transfer characteristics. In various embodiments, the propylene glycol concentration ranges from approximately 20% to approximately 80% by volume, with concentrations of approximately 30% to approximately 70% by volume providing particularly favorable characteristics for patient temperature management applications. In some embodiments, the propylene glycol concentration is 50% by volume.

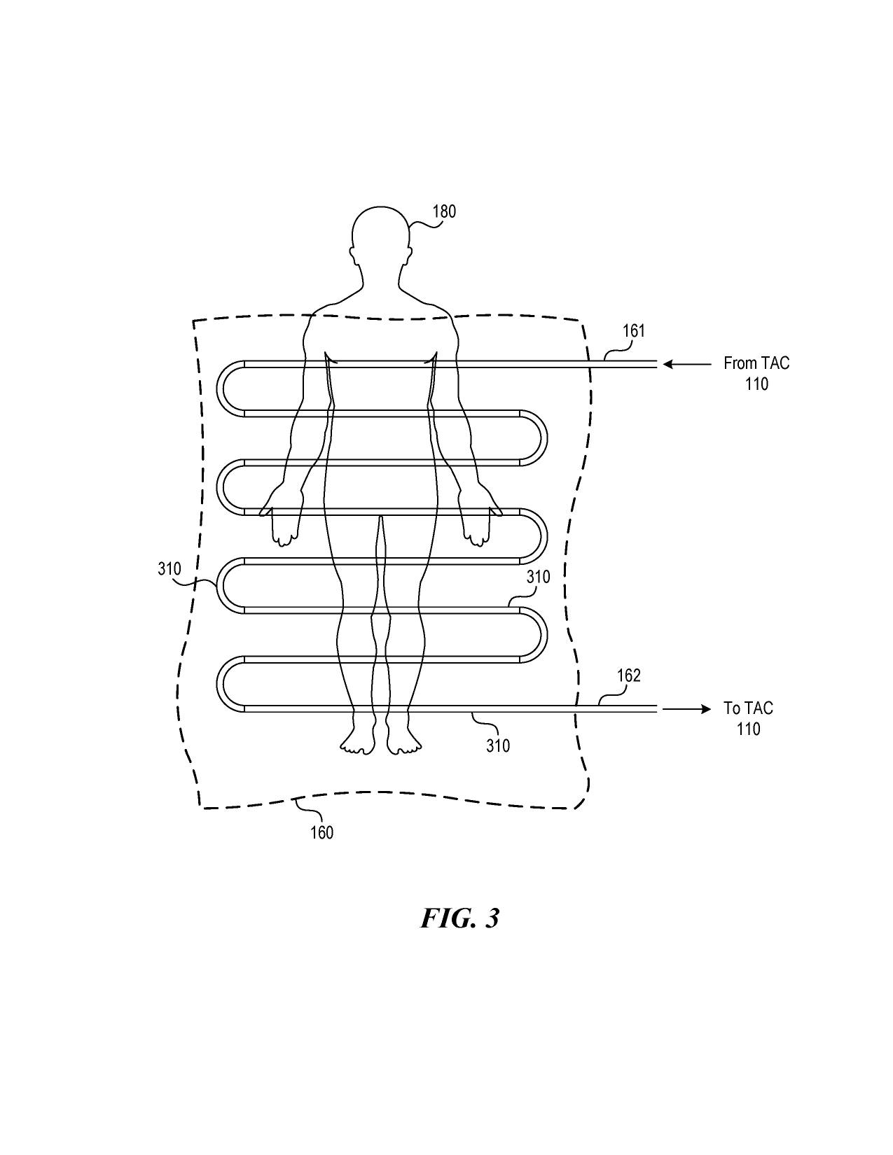

FIG. 3 illustrates in greater detail the thermal blanket 160 of FIG. 2 according to embodiments of the present technology. As shown, the thermal blanket 160 comprises a flexible hose 310 that circulates the thermal liquid throughout the interior volume of the thermal blanket 160. The first end of the flexible hose 310 couples to the input conduit 161 and receives the incoming thermal liquid from the TAC 110. The second end of the flexible hose 310 couples to the output conduit 162 and transmits the outgoing thermal liquid back to the TAC 110.

The use of flexible hose 310 is merely an example of a system for circulating the heating/cooling thermal liquid throughout the thermal blanket 160. In alternate embodiments, the interior of the thermal blanket 160 comprises a series of small, connected cavities or chambers that form a honeycomb-like pattern or other diverting and converging pattern. In such embodiments, an inlet port of the thermal blanket 160 couples to the input conduit 161 to receive incoming thermal liquid. At the other end of the thermal blanket 160, an outlet port couples to the output conduit 162.

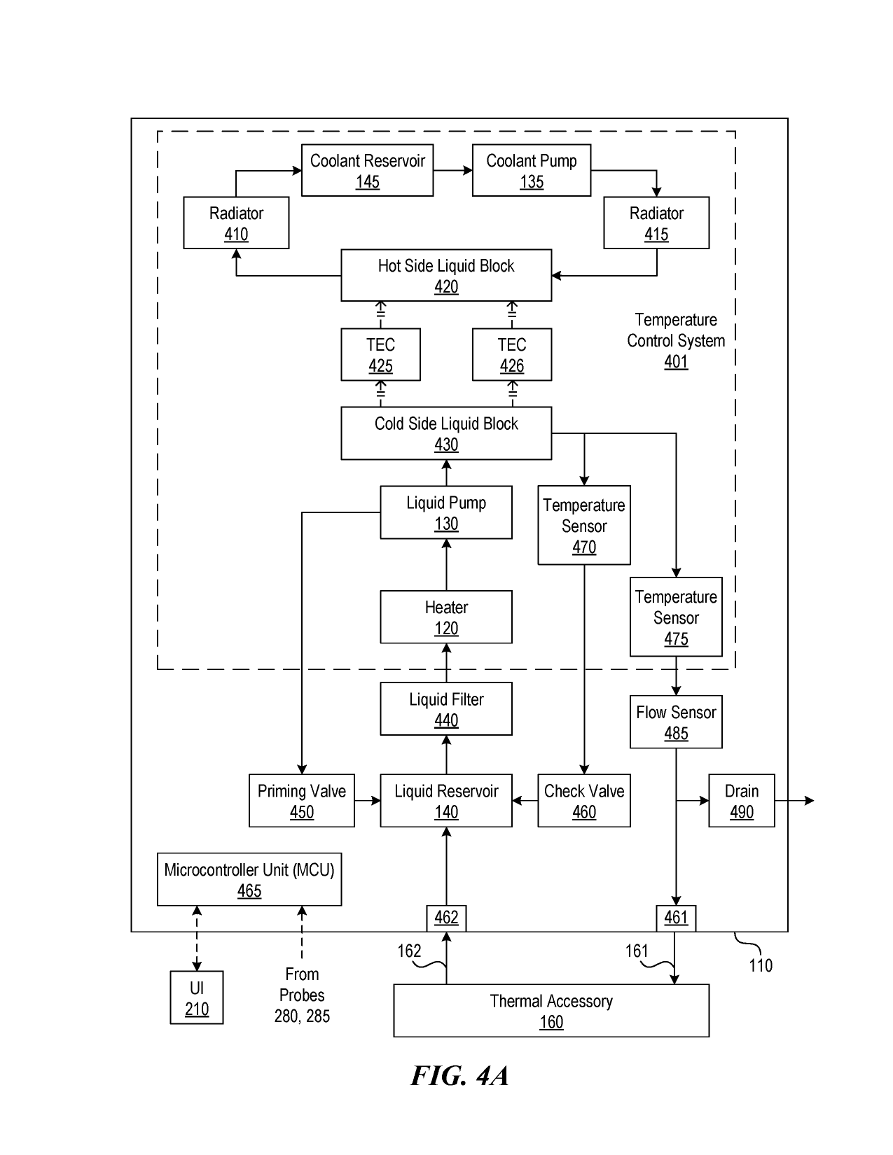

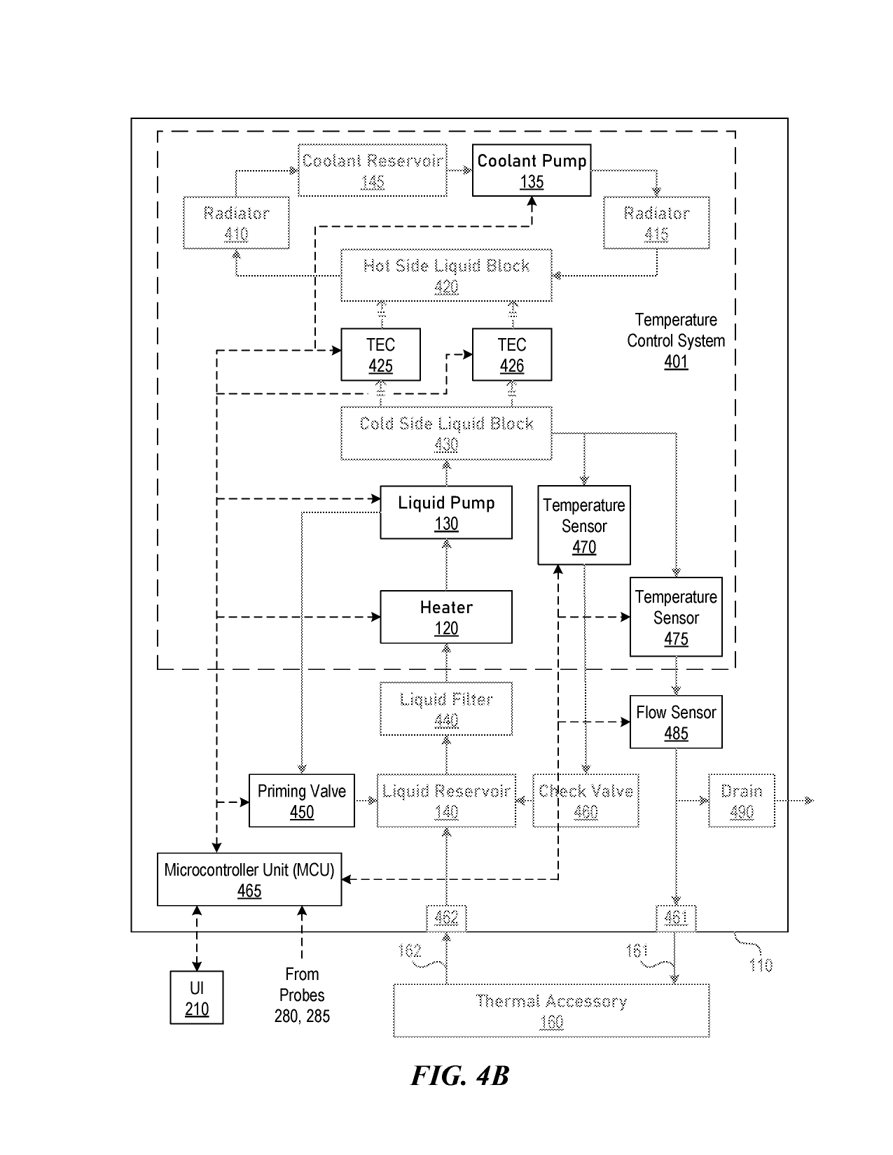

FIG. 4A is a block diagram illustrating a fluid path of the TAC 110 of FIG. 1. As shown, the TAC 110 includes a temperature control system (TCS) 401, shown in a dotted outline. The TCS 401 comprises the heater 120, the liquid pump 130, the coolant pump 135, and the coolant reservoir 145 described above with respect to FIG. 1 as well as temperature sensors 470 and 475, cold side liquid block 430, TEC 425 and TEC 426, hot side liquid block 420, radiator 410, and radiator 415. The TAC 110 also includes the liquid reservoir 140 described above with respect to FIG. 1 as well as a microcontroller unit (MCU) 465, a priming valve 450, a liquid filter 440, a flow sensor 485, a check valve 460, a drain 490, an output port 461, and an input port 462. As shown, the TAC 110 interfaces with thermal accessory 160, the input conduit 161, and the output conduit 162 described above with respect to FIG. 1 as well as the UI 210 and the temperature probes 280 and 285 described above with respect to FIG. 2. Each of these components can be selected to accommodate a glycol-based thermal liquid (e.g., as described above with respect to FIG. 1).

The fluid path of the TAC 110 is illustrated by solid arrow lines. Communication between components of the TAC 110 is illustrated by dotted arrow lines. Thermal transfer between components of the TAC 110 is illustrated by broken solid arrow lines. As shown, thermal liquid returning from the thermal accessory 160 enters the TAC 110 through the input port 462. The input port 462 is a fluid inlet configured to receive thermal liquid from an external thermal accessory (e.g., via the output conduit 162 of FIG. 1). In various embodiments, the input port 462 comprises a quick-connect fitting, a barbed connector, a threaded coupling, a luer lock fitting, or other suitable fluid connection mechanism. In some embodiments, the input port 462 includes a passive valve (e.g., a spring and plunger) that is configured to open in response to a force applied to the passive valve when the input port 462 is connected to the output conduit 162. In other embodiments, the input port 462 represents one of multiple input ports 462 (e.g., 2, 3, or more input ports) that each receive thermal liquid from a different external thermal accessory and flow the thermal liquid into the liquid reservoir 140 as described below.

From the input port 462, the thermal liquid flows into the liquid reservoir 140. As described above with respect to FIG. 1, the liquid reservoir 140 is a storage vessel configured to hold a volume of thermal liquid. In the disclosed configuration, the liquid reservoir 140 is positioned in continuous fluid communication with the main fluid path of the TAC 110. This is expected to provide a significant advantage over conventional patient temperature management systems, which typically incorporate a cold water or ice tank as a separate reservoir in a parallel arrangement that is only accessed when patient cooling is required. By inserting the liquid reservoir 140 in continuous fluid communication with the circulating thermal liquid of the TAC 110, the present technology can leverage the thermal mass of the liquid reservoir 140 to stabilize the circulating fluid's temperature. The substantial thermal mass of the liquid reservoir 140 acts as a thermal buffer, absorbing transient heat inputs or losses without experiencing large temperature swings, thereby dampening temperature fluctuations caused by variations in heat load, environmental conditions, or intermittent operation of heating and cooling elements.

From the liquid reservoir 140, the thermal liquid flows through the liquid filter 440. The liquid filter 440 is a filtration device configured to remove particulates, debris, and contaminants from the circulating thermal liquid. In various embodiments, the liquid filter 440 comprises a mesh screen filter, a pleated filter element, a depth filter, a membrane filter, a cartridge filter, or other suitable filtration mechanism capable of maintaining fluid cleanliness within the TAC 110.

After passing through the liquid filter 440, the thermal liquid passes through the heater 120. The heater 120 is configured to add thermal energy to the circulating thermal liquid when the TAC 110 is operating in heating mode. The MCU 465 (described in further detail below) selectively activates (also referred to herein as “controllably activates” or “dynamically activates”) the heater 120 to raise the temperature of the thermal liquid to a target setpoint. The MCU 465 regulates the power delivered to the heater 120 based on feedback from temperature sensors within the fluid path, enabling precise control of the thermal liquid temperature. For example, the MCU 465 can receive temperature data from the temperature sensors 470 and 475, compare the temperature data to a threshold temperature value, and activate the heater 120 based on the difference between the temperature data and the threshold temperature value. In various embodiments, the heater 120 is capable of heating the thermal liquid to temperatures suitable for patient warming applications, including temperatures up to approximately 70°C for high-temperature disinfection procedures. The heater 120 may operate at varying power levels as determined by the MCU 465 to achieve rapid heating when a large temperature differential exists between the current liquid temperature and the target temperature, or to maintain a stable temperature when the thermal liquid is near the target setpoint.

After passing through the heater 120, the thermal liquid enters the liquid pump 130. The liquid pump 130 draws the thermal liquid from the liquid reservoir 140 (through the liquid filter 440 and the heater 120) and propels it through the remainder of the fluid path. The liquid pump 130 is selected to accommodate the increased viscosity characteristics of glycol-based thermal liquids compared to pure water. In various embodiments, the liquid pump 130 includes a tachometer configured to measure the rotational speed of the pump motor. The tachometer transmits speed measurement signals to the MCU 465, which monitors the pump speed and adjusts the motor speed as needed to ensure that the glycol-based thermal liquid circulates at a flow rate comparable to that which would be achieved with water. This speed compensation accounts for the higher viscosity of the glycol-water mixture, which would otherwise result in reduced flow rates for a given pump speed setting. By dynamically adjusting the motor speed based on tachometer feedback, the MCU 465 maintains consistent thermal transfer performance regardless of variations in fluid viscosity caused by temperature changes or differences in glycol concentration.

The priming valve 450 is connected to the liquid reservoir 140 to facilitate priming operations. The priming valve 450 is a valve mechanism configured to enable removal of air from the liquid pump 130 and associated fluid pathways during initial system setup or after maintenance procedures. In various embodiments, the priming valve 450 comprises a manual bleed valve, an automatic air vent valve, a solenoid-actuated valve, a ball valve, or other suitable valve mechanism. The inclusion of the priming valve 450 addresses a shortcoming of conventional liquid-circulating patient temperature management systems, which often require trapped air to escape passively through a tortuous water path during initial fluid priming. Such priming configurations slow the priming process for the pump and may not achieve complete air removal, leading to potential damage.

The priming valve 450, on the other hand, is expected to enable efficient priming of the liquid pump 130 , reducing the risk of residual air pockets that can compromise pump performance by causing cavitation, flow interruptions, or inconsistent pressure delivery. More specifically, in some embodiments, the priming valve 450 is disposed close to the liquid pump 130. This provides a low-resistance pathway for air to quickly evacuate when the liquid pump 130 is initially flooded with liquid, enabling quicker priming and preventing device damage. Generally, during priming, the priming valve 450 is temporarily opened to allow the liquid pump 130 to evacuate air and then shuts once the liquid pump 130 is flooded with fluid so that air can be evacuated from the rest of the fluid path. As described above, in conventional systems, there is no dedicated line or valve for priming a pump. Instead, air must escape through the fluid path, which can make priming slow, difficult, and, depending on the system, could damage the device over time.

The liquid pump 130 pumps the thermal liquid into the cold side liquid block 430. The cold side liquid block 430 is a heat transfer component configured to facilitate thermal energy exchange between the circulating thermal liquid and the thermoelectric coolers. In various embodiments, the cold side liquid block 430 comprises an aluminum block with machined passages (i.e., internal fluid channels), a copper block with machined passages, a liquid cold plate, a microchannel heat sink, or other suitable heat transfer device. In some embodiments, the cold side liquid block 430 includes an internal channel for the circulating thermal liquid, thereby allowing heat to transfer between the circulating thermal liquid and the bulk metal of the cold side liquid block 430.

In embodiments that utilize a glycol-based thermal liquid, the cold side liquid block 430 can be cooled to temperatures below 0°C without risk of ice formation or fluid solidification within the internal channels. The freezing point depression provided by the glycol concentration enables the thermal liquid to remain in liquid phase at sub-zero temperatures, allowing the cold side liquid block 430 to achieve temperatures that would cause pure water to freeze and obstruct flow. This capability provides several advantages for patient cooling applications. First, the ability to operate the cold side liquid block 430 at sub-zero temperatures increases the temperature differential available for heat transfer, enabling more rapid cooling of the thermal liquid when aggressive patient cooling is required. Second, the extended low-temperature operating range provides a greater margin of safety during cooling procedures, as the system can continue to function even if temperatures temporarily drop below the freezing point of water due to control overshoot or transient conditions. Third, the sub-zero capability enables the TAC 110 to pre-cool the thermal liquid to lower temperatures before connecting a thermal accessory, reducing the time required to achieve target patient temperatures in time-sensitive clinical scenarios such as targeted temperature management following cardiac arrest.

The TEC 425 and TEC 426 are positioned between the cold side liquid block 430 and the hot side liquid block 420. In some embodiments, each of the TEC 425 and the TEC 426 comprise two double-stacked TECs positioned laterally to each other (i.e., 4 TECs split into stacked pairs positioned next to one another). In other embodiments, each of the TEC 425 and the TEC 426 are single double-stacked TECs. Still further, the present technology envisions the use of any number of TECs of any form. For example, the TAC 110 may include a single TEC that is not double-stacked, 2 TECs that have varying stack compositions, 4 TECs, 10 TECs, and so on.

The use of thermoelectric coolers provides a significant advantage over conventional patient temperature management systems for non-surgical applications, which rely on refrigeration-based chiller blocks for cooling functionality. Refrigeration-based chillers are typically large and heavy, creating logistical difficulties in healthcare settings where floor space is limited. Additionally, refrigerants introduce safety hazards and environmental concerns related to leakage or disposal, and require specialized assembly processes such as brazing of refrigerant lines. By replacing refrigeration-based cooling with thermoelectric coolers, the disclosed system achieves a more compact equipment footprint and ease of deployment in space-constrained treatment areas.

The TEC 425 and TEC 426 operate under the control of the MCU 465 to cool the thermal liquid flowing within the cold side liquid block 430. When the MCU 465 activates the thermoelectric coolers, an electrical current passes through the semiconductor junctions within the TEC 425 and TEC 426, causing heat to be transferred from the cold side to the hot side via the Peltier effect. The MCU 465 (described in further detail below) selectively activates (also referred to herein as “controllably activates” or “dynamically activates”) the TECs to lower the temperature of the thermal liquid to a target setpoint. For example, the MCU 465 can receive temperature data from the temperature sensor 475, compare the temperature data to a threshold temperature value, and activate the TECs based on the difference between the temperature data and the threshold temperature value.

Heat transfer is illustrated in FIG. 4A by the broken solid arrow lines between the cold side liquid block 430, the TECs 425 and 426, and the hot side liquid block 420. The cold surfaces of the TEC 425 and TEC 426 are in thermal contact with the cold side liquid block 430, thereby absorbing thermal energy from the bulk metal of the cold side liquid block 430 and, consequently, from the thermal liquid flowing through its internal channels. Though the embodiment of FIG. 4A includes two TECs, other embodiments include a multiplicity of thermoelectric coolers that are positioned next to one another or stacked on top of one another.

The hot side liquid block 420 is a heat transfer component positioned on the opposite side of the TEC 425 and TEC 426 from the cold side liquid block 430. The hot side liquid block 420 is configured to receive and dissipate the waste heat generated by the thermoelectric cooling process. In various embodiments, the hot side liquid block 420 comprises an aluminum block with machined passages (i.e., internal fluid channels), a copper block with machined passages, or other suitable heat transfer device capable of efficiently transferring heat from the hot surfaces of the TEC 425 and TEC 426 to a circulating coolant liquid.

In some embodiments, an additional temperature sensor is attached to the hot side liquid block 420. The additional temperature sensor transmits temperature measurements to the MCU 465. When the additional temperature sensor measures a temperature above a particular threshold, the MCU 465 can turn off power to the TEC 425 and the TEC 426. In doing so, the MCU 465 can protect the hot side liquid block 420 from getting too hot (which may damage the TECs, cause excess heating of the thermal liquid, cause a fire, etc.). In some embodiments, the MCU 465 can identify that a failure in the coolant loop may have occurred based on measurements from the additional temperature sensor.

The coolant pump 135 circulates coolant liquid through a heat dissipation circuit that includes the hot side liquid block 420, the radiator 410, the radiator 415, and the coolant reservoir 145. The coolant pump 135 draws coolant liquid from the coolant reservoir 145 and propels it through the radiator 415 and the hot side liquid block 420, where the coolant liquid absorbs thermal energy from the bulk metal of the hot side liquid block 420. The heated coolant liquid then flows through the radiator 410 and returns to the coolant reservoir 145. The radiator 410 and the radiator 415 are heat dissipation devices configured to transfer thermal energy from the coolant liquid to the surrounding environment. In various embodiments, the radiator 410 and the radiator 415 comprise finned heat exchangers, fan-cooled radiators, or other suitable heat dissipation mechanisms. The coolant reservoir 145 provides a volume of coolant liquid that adds thermal mass to the heat dissipation circuit and accommodates thermal expansion of the coolant liquid during operation. In various embodiments, the coolant liquid comprises water, a water-glycol mixture, a water-propylene glycol solution, a water-ethylene glycol solution, deionized water, or other suitable heat transfer fluid.

The effectiveness of the thermoelectric cooling provided by the TEC 425 and TEC 426 is directly related to the temperature differential between the hot side and cold side of the thermoelectric coolers. By actively removing heat from the hot side liquid block 420 through the coolant circulation system, the temperature of the hot side liquid block 420 is maintained at a lower level than would otherwise be achievable through passive cooling alone. This reduction in hot side temperature is expected to enable the TEC 425 and TEC 426 to achieve a greater temperature differential across their semiconductor junctions, resulting in a correspondingly lower temperature on the cold side liquid block 430. Accordingly, the coolant circulation system comprising the coolant pump 135, the hot side liquid block 420, the radiator 410, the radiator 415, and the coolant reservoir 145 enhances the cooling capacity of the thermoelectric coolers and enables the TAC 110 to achieve lower thermal liquid temperatures for patient cooling applications.

From the cold side liquid block 430, the thermal liquid may follow one of two portions of the fluid path of the TAC 110 depending on whether a thermal accessory is attached to the TAC 110. The first portion of the fluid path – used when no thermal accessory is attached – includes the temperature sensor 470 and the check valve 460. The temperature sensor 470 and the check valve 460 can be on either side of one another. As such, the first portion of the fluid path may first flow the thermal liquid through the check valve 460 and then past the temperature sensor 470, or vice versa. When no thermal accessory is attached, the thermal liquid flows from the cold side liquid block 430 through the check valve 460 and then through temperature sensor 470. The check valve 460 is a passive valve mechanism that allows the thermal liquid to return to the liquid reservoir 140 when a thermal accessory is not connected. This configuration addresses a significant shortcoming of conventional liquid-circulating patient temperature management systems, which encounter issues when a thermal accessory is disconnected. In conventional systems, the flow path is interrupted when a thermal accessory is removed, leaving the circulating pump with nowhere for the fluid to go, resulting in pressure buildup that can damage system components or disrupt operation. The check valve 460 enables continuous fluid circulation through the TAC 110 even when no thermal accessory is attached, reducing the risk of pressure buildups. The continuous fluid circulation using the check valve 460 also permits pre-chilling or pre-warming of the thermal liquid as well as monitoring of fluid temperatures during accessory changeover periods. In various embodiments, the check valve 460 comprises a spring-loaded check valve, a ball check valve, a swing check valve, a diaphragm check valve, or other suitable one-way valve mechanism.

The second portion of the fluid path – used when a thermal accessory is attached – includes the flow sensor 485 and the temperature sensor 475. The temperature sensor 475 and the flow sensor 485 can be on either side of one another. As such, the second portion of the fluid path may first flow the thermal liquid through the flow sensor 485 and then past the temperature sensor 475, or vice versa. The flow sensor 485 is a flow measurement device configured to monitor the volumetric flow rate of the thermal liquid through the second portion of the fluid path. In some embodiments, the MCU 465 uses flow rate data from the flow sensor 485 to adjust the speed of the liquid pump 130 when a glycol-based thermal liquid is circulating through the system, compensating for the increased viscosity of the glycol solution by increasing pump speed to achieve a flow rate comparable to that which would be achieved with water.

When a thermal accessory is attached, the thermal liquid flows from the cold side liquid block 430 through the flow sensor 485 and the temperature sensor 475, then exits the TAC 110 to the thermal accessory 160 via the output port 461. The output port 461 is a fluid outlet configured to deliver temperature-controlled thermal liquid to an external thermal accessory. In various embodiments, the output port 461 comprises a quick-connect fitting, a barbed connector, a threaded coupling, a luer lock fitting, or other suitable fluid connection mechanism. The output port 461 couples to the input conduit 161, which carries the thermal liquid to the thermal accessory 160. In some embodiments, the output port 461 includes a passive valve (e.g., a spring and plunger) that is configured to open in response to a force applied to the passive valve when the output port 461 is connected to the input conduit 161. In other embodiments, the output port 461 represents one of multiple output ports 461 (e.g., 2, 3, or more output ports) that each carry the thermal liquid to a different external thermal accessory. In yet further embodiments, the drain 490 is also included in this second portion of the fluid path to allow drainage of the system during maintenance or fluid replacement procedures. In various embodiments, the drain 490 comprises a drain valve, a drain port, a petcock, or other suitable drainage mechanism that enables removal of thermal liquid from the TAC 110.

The temperature sensor 470 and temperature sensor 475 are temperature sensing devices configured to measure the temperature of the thermal liquid at various points in the fluid path (e.g., at the first and second portions of the fluid path). In various embodiments, the temperature sensors 470 and 475 comprise thermistors, resistance temperature detectors (RTDs), thermocouples, integrated circuit temperature sensors, or other suitable temperature measurement devices. The flow sensor 485 is a flow measurement device configured to monitor the volumetric or mass flow rate of the thermal liquid through the fluid path (e.g., through the second portion of the fluid path). In various embodiments, the flow sensor 485 comprises an ultrasonic flow sensor, a turbine flow meter, a magnetic flow meter, a differential pressure flow sensor, or other suitable flow measurement device.

In some embodiments, even when a thermal accessory is attached to the TAC 110 and the majority of the thermal liquid flows through the second portion of fluid path, a small amount of thermal liquid will continue to flow through the first portion of the fluid path. Accordingly, temperature readings from the temperature sensor 470 in the first portion of the fluid path remain valuable for monitoring purposes, as they provide additional temperature data that can be used by the microcontroller unit 465 to verify system operation, enhance temperature control accuracy, and ensure patient safety.

The MCU 465 controls the overall operation of the TAC 110. In one embodiment, while in heating mode, the MCU 465 causes the liquid pump 130 to pump the temperature-controlled thermal liquid from the liquid reservoir 140 through the liquid filter 440, through the heater 120, and into the liquid pump 130. The liquid pump 130 then pumps the thermal liquid into the cold side liquid block 430, through one or more sensors 475 and 485, and then out of the TAC 110 via the output port 461 and input conduit 161 of the thermal accessory 160. The returning thermal liquid re-enters the TAC 110 via the output conduit 162 of the thermal accessory 160 and the input port 462, and then flows back into the liquid reservoir 140.

In heating mode, the heater 120 is active. The TEC 425 and TEC 426 are inactive, as are the hot side liquid block 420, the radiators 410 and 415, the coolant pump 135, and the coolant reservoir 145. In cooling mode, the MCU 465 deactivates the heater 120, and the liquid pump 130 continues to circulate the thermal liquid through the cold side liquid block 430 while the TEC 425 and TEC 426 are turned ON and operating. The cold side liquid block 430 includes an internal channel for the circulating thermal liquid, thereby allowing heat to transfer from the circulating thermal liquid to the bulk metal of the cold side liquid block 430. In some embodiments, the TEC 425 and TEC 426 cool the cold side liquid block 430 by transferring heat from the bulk metal of the cold side liquid block 430 to the bulk metal of the hot side liquid block 420 by means of the Peltier effect. In the Peltier effect, an electrical voltage is applied across an electronic part to move heat from one side of the TEC to the other side, thereby creating separate cold and hot surfaces.

In some embodiments, under control of the UI 210, the MCU 465 executes a set of user functions that include a run function, a test function, a prime function, a clean function, a settings function, and a maintenance function. The run function sets, controls, and monitors both the patient 180 and the internal liquid temperature of the TAC 110. There are three main modes within the run function: i) a manual mode, ii) a probe mode, and iii) a smart mode.

The manual mode allows an operator to control the thermal liquid temperature that is output to the thermal blanket 160. The probe mode allows an operator to monitor temperature probes 280 and 285 as well as set the desired patient temperature. An operator can see the liquid temperature of the internal circuit of the TAC 110 while having control of the patient temperature directly. The smart mode uses the same approach as the probe mode with the added feature of allowing an operator to select the maximum temperature differential (also referred to herein as a “max differential”) between the patient and the thermal liquid in the thermal accessory 160.

In smart mode, the TAC 110 requests a user-desired patient temperature, instead of a fluid temperature. The TAC 110 then uses the external temperature probes 280 and 285 as feedback and activates the heater and cooler as needed to cause the patient temperature to reach the user-desired temperature. During smart mode, the user also enters the max differential. Generally, this is a safety feature corresponding to a maximum difference the TAC 110 can allow between the measured patient's current temperature and the measured thermal fluid temperature. For example, if the user is heating the patient to some temperature and the max differential is 5.0 °C, then the TAC 110 cannot allow the fluid temperature to exceed the patient's current temperature + 5.0 °C while it is heating them.

In some embodiments, the smart mode includes a smart assist setting. If the smart assist setting is enabled, the TAC 110 can automatically determine whether the user selected max differential is sufficient to overcome the thermal intertia of the patient and meaningfully change the patient’s temperature. If the max differential is insufficient, the TAC 110 will automatically apply an offset to the max differential until the patient starts meaningfully changing in temperature in the desired direction. The TAC 110 will then reduce or remove the offset.

The test function of the TAC 110 allows an operator to run an internal check on the functionality of the internal components of the TAC 110 and provides visual feedback on their status (pass/fail). For a pump to work properly, the liquid path of the pump must be fully flooded and free of any air in the system. The prime function ensures the internal liquid path of the TAC 110 is flooded with thermal transfer liquid and that air is taken out of the system. In some embodiments, the prime function employs the priming valve 450 to ensure that the internal liquid path of the TAC 110 is flooded with thermal transfer liquid and that air is taken out of the system.

The clean function of the TAC 110 directs an operator through a cleaning and disinfection process of the TAC 110 and allows the TAC 110 to perform the necessary internal cycles for a successful cleaning and disinfection. In embodiments that utilize a glycol-based thermal liquid, the clean function leverages the elevated boiling point of the glycol-water mixture to implement a high-temperature disinfection procedure. During the high-temperature disinfection procedure, the TAC 110 circulates the glycol-based thermal liquid at temperatures of approximately 70°C or higher for a predetermined duration sufficient to achieve effective microbial decontamination. The elevated boiling point of the glycol-based mixture, which exceeds 100°C depending on the glycol concentration, may reduce the risk that the thermal liquid leaves the liquid phase throughout the disinfection procedure. Additionally, the inherent antimicrobial properties of the glycol-based thermal liquid complement the thermal disinfection process by inhibiting microbial growth between cleaning cycles, thereby reducing the overall microbial load within the fluid pathways and extending the intervals between required cleaning and disinfection procedures. The clean function may also include prompts for the operator to connect a loop back connector to the TAC 110, enabling the thermal liquid to circulate through all internal plumbing components during the disinfection procedure without requiring a thermal accessory to be attached.

The settings function of the TAC 110 allows an operator to view and customize various features of the TAC 110, such as screen brightness, screen sound, language, device name, and enabling or disabling a low energy consumption mode.

The maintenance function of the TAC 110 allows an operator to see pertinent contact information for when it is necessary to service the TAC 110. Additionally, maintenance mode provides an operator with system information regarding the software version, the user interface version, and the number of hours the TAC 110 has been in operation. The maintenance function also provides an operator with a password-protected technician portal in which the operator may access more in-depth information about the past operation of the device, such as error logs, cleaning and disinfection treatments performed on the device, and the like.

In an example embodiment, the TAC 110 controls liquid temperature by regulating the power to heater 120, which may be, for example, a 1200-watt stainless steel heater chamber. The temperature of the liquid is set by the operator through the screen of UI 210. Once the liquid temperature is set, the system regulates the power to the heater 120 to control the liquid temperature. The power to the heater 120 is regulated through a closed-loop feedback system which receives input data from the temperature probes 280 and 285 and temperature sensors 470 and 475.

FIG. 4B is a block diagram illustrating data channels of a patient temperature management system according to various aspects of the present technology. Each of the components of FIG. 4B are described further above with respect to FIG. 4A. As shown here, the MCU 465 is in communication with the UI 210, the temperature probes 280 and 285, the heater 120, the liquid pump 130, the coolant pump 135, the TECs 425 and 426, the priming valve 450, the temperature sensors 470 and 475, and the flow sensor 485. Generally, the MCU 465 is in electrical communication with each of these components through dedicated signal lines, data buses, or control interfaces. However, across various embodiments, the MCU 465 interfaces with these components through wireless communication protocols.

The MCU 465 can communicate with the UI 210 to receive operator commands and setpoint values from the UI 210 and to transmit status information, temperature readings, and alarm conditions to the UI 210 for display to the operator. In some embodiments, the MCU 465 receives analog or digital temperature signals from the temperature probes 280 and 285. In embodiments where the temperature probes 280 and 285 comprise thermistors or RTDs, the MCU 465 includes analog-to-digital converter (ADC) circuitry to convert the resistance-based temperature signals into digital values for processing. Similarly, the MCU 465 can receive temperature data from the temperature sensors 470 and 475 through analog or digital communication interfaces depending on the sensor type.

The MCU 465 controls the heater 120 by transmitting signals that regulate the power delivered to the heating element. For example, the MCU 465 may output a pulse width modulation (PWM) signal to a solid-state relay, a triac, or a phase control device that modulates the AC power supplied to the heater 120, thereby controlling the heating rate. The MCU 465 controls the liquid pump 130 and the coolant pump 135 through motor driver circuits that receive speed control signals from the MCU 465. In embodiments where the pumps comprise brushless DC motors, the MCU 465 may transmit PWM signals to electronic speed controllers that regulate pump speed. The MCU 465 may also receive feedback signals from the pumps indicating rotational speed or operational status.

The MCU 465 controls the TECs 425 and 426 by transmitting control signals to power driver circuits that regulate the electrical current supplied to the TECs. The MCU 465 may output PWM signals or analog voltage signals that determine the cooling power delivered by the TECs 425 and 426. The MCU 465 controls the priming valve 450 through a digital output signal that actuates a solenoid or motor within the valve mechanism. For example, the MCU 465 may set a digital output pin high to open the priming valve 450 during priming operations and set the pin low to close the valve during normal operation. Finally, the MCU 465 receives flow rate data from the flow sensor 485 through a digital pulse output, an analog voltage signal, or a digital communication interface depending on the sensor type. For example, in embodiments where the flow sensor 485 comprises a turbine flow meter, the MCU 465 may count pulses from the sensor to determine the volumetric flow rate of the thermal liquid.

In some embodiments, the TAC 110 includes an alarm system. Such an alarm system can include a separate circuit board that supports an alarm (e.g., an alarm buzzer). The alarm can produce sound volumes, frequencies, and intervals that comply with medical safety standards for such alarms. The separate circuit board can be in data communication with the MCU 465. If the MCU 465 detects faults or anomalous sensor readings, the MCU 465 can cause the alarm to activate with a sound corresponding to a priority of the fault or anomaly.

The separate circuit board and the alarm can be battery-powered. Additionally, the alarm system can be configured to monitor the state of the main power provided to the TAC 110 as well as, or alternatively, the state of the main power switch. If the TAC 110 loses power, but its power switch is in the ON position, the alarm system can automatically activate to alert a user to a power failure.

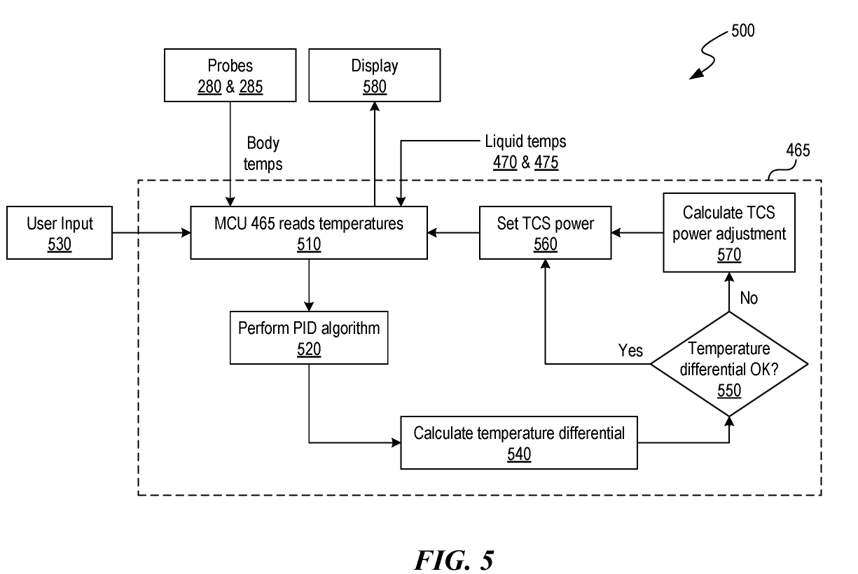

FIG. 5 is a flow diagram 500 illustrating the operation of the microcontroller unit (MCU) 465 according to one embodiment of the disclosure. In 510, the MCU 465 reads body temperature data from the temperature probes 280 and 285, liquid temperature data from the temperature sensors 470 and 475, and/or a user input 530. In 520, MCU 465 acts as a proportional-integral-derivative (PID) controller and performs a PID control algorithm. A PID controller continuously calculates an error value as the difference between a target setpoint and a measured process variable (PV) (i.e., the input from 510) and applies a correction based on PID terms. The PID algorithm automatically applies an accurate and responsive correction with minimal delay and overshoot.

In 540, the MCU 465 calculates the magnitude of a temperature differential between the target temperature and the measured body temperature. In 550, the MCU 465 determines if the magnitude of the temperature difference is less than a threshold value representing the safe allowable temperature differential between the circulating thermal liquid temperature and the body temperature (i.e., a first threshold value which could be set by MCU 465 or set by the operator in smart mode). If Yes in 550, the MCU 465 in 560 maintains the power the TCS 401 provides to the heater 120 and to the TECs 425 and 426 and returns to 510. If the magnitude of the temperature difference exceeds the threshold value (No in 550), the MCU in 570 calculates an adjustment that the TCS 401 makes to the power applied to the heater 120 and to the TECs 425 and 426. The MCU 465 then sets the TCS power to the new level in 560 and returns to 510.

In some embodiments, the MCU 465 outputs temperature and status information to a display 580 for visualization by an operator. The display may be integrated into the UI 210 or may comprise a separate display device coupled to the TAC 110. Across various embodiments, the display presents real-time temperature readings from the temperature probes 280 and 285, liquid temperature readings from the temperature sensors 470 and 475, flow rate information from the flow sensor 485, and operational status indicators for the various components of the TAC 110. In other embodiments, the display comprises a liquid crystal display (LCD), a light-emitting diode (LED) display, an organic light-emitting diode (OLED) display, a touchscreen display, or other suitable visual output device.

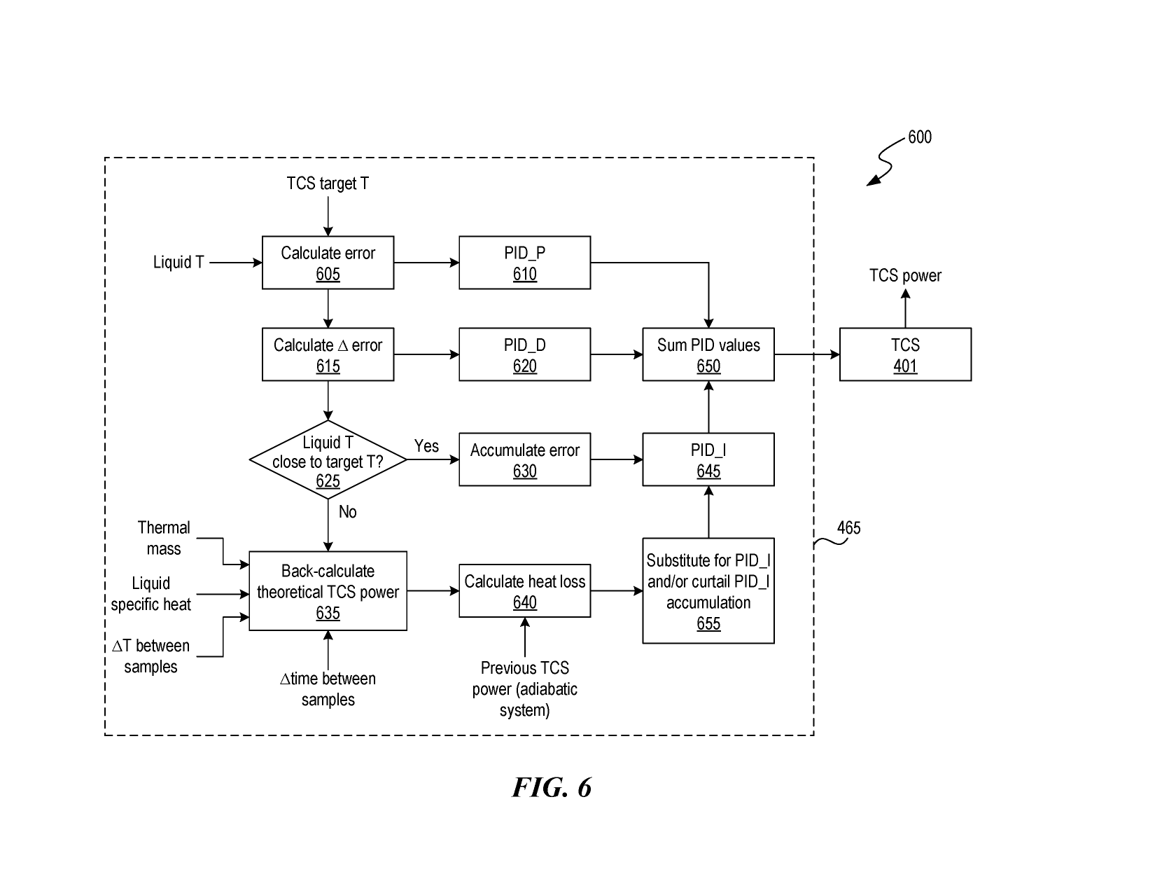

FIG. 6 is a flow diagram 600 illustrating the operation of the proportional-integral-differential (PID) algorithm performed by the microcontroller unit (MCU) 465 in the TAC 110 according to one embodiment of the disclosure. The MCU 465 executes a PID algorithm that employs a novel technique that compares the thermal losses in the patient thermal regulation system 200 to an adiabatic system (i.e., a system in which no heat transfer occurs) to balance more accurately a variety of thermal loads.

In the disclosed PID algorithm, the MCU 465 calculates the proportional component (PID_P) and the derivative component (PID_D) as normal. However, the MCU 465 calculates the integral component (PID_I) differently, depending on the magnitude of the difference between the TCS target temperature, TCS(T), and the current temperature of the thermal liquid, Liquid(T). When the target temperature is very close to the current temperature, the MCU 465 calculates the PID_I component as expected (i.e., by continually adding the error to an accumulating variable). When the target temperature is not close to the current temperature, the MCU 465 modifies the PID_I component by substitution with a value representative of the heat dissipated by the patient thermal regulation system 200, by curtailing the magnitude (i.e., absolute value) of the maximum allowable accumulating PID_I variable with a value representative of the heat dissipated by the patient thermal regulation system 200, or a combination thereof.

The MCU 465 determines the replacement value by comparing the actual heat energy added to the TCS 401 to the heat energy added to an adiabatic system. The energy added in an adiabatic system (i.e., no thermal losses) is simply the set value of power for the TCS 401. Using a previously stored temperature value at a known point in time, the thermal energy added to the TCS 401 is back calculated using standard thermodynamic equations. The difference between these two values is a good estimate for the heat lost by the TCS 401. The accumulating variable PID_I is then set to the replacement value or curtailed by the replacement value. The unique method of processing the PID_I component provides a novel control mechanism that back calculates the thermal energy lost in the TCS 401 system to obtain an estimated balancing point before the system temperature reaches a target temperature.

The disclosed PID algorithm prevents the overshooting, undershooting, and oscillation that would normally occur in a traditional PID control system, since under normal circumstances, the PID_I component would be accumulating to arbitrarily large positive or negative values while the TCS 401 is adjusting the temperature of the system thermal liquid. Due to the slow response time of a TCS 401 when heating or cooling a large volume of thermal liquid, the PID algorithm is necessary to obtain accurate temperature balancing at a variety of thermal loads and losses.

In FIG. 6, the MCU 465 calculates in 605 an error value (or difference) between the target temperature, TCS(T) of the TCS 401, and the current thermal liquid temperature, Liquid(T). The TCS(T) – Liquid(T) error value is the input of the proportional PID_P calculation controller 610. Thus, the output of PID_P calculation controller 610 is proportional to the current value of the TCS(T) – Liquid(T) error value. For example, if the error value is large and positive, the control output of PID_P calculation controller 610 is proportionately large and positive according to a gain factor “K”.

In 615, the MCU 465 calculates the rate of change (or Δ) in the error value. The Δ error is the input of the PID_D calculation controller 620. The output of the PID_D calculation controller 620 comprises a best estimate of the future trend of the TCS(T) – Liquid(T) error value, based on its current rate of change. The more rapid the change, the greater the controlling or damping effect by the PID_D calculation controller 620.

In 625, the MCU 465 determines if the thermal liquid temperature Liquid(T) is close to the target temperature TCS(T) by comparing the difference between the thermal liquid temperature Liquid(T) and the target temperature TCS(T) to a second threshold value representing a shift from TCS function from a temperature adjustment mode to a temperature balancing mode. If Yes in 625, the MCU 465 in 630 calculates the PID_I component normally by accumulating the past values of the TCS(T) – Liquid(T) error value, which forms the input to the PID_I calculation controller 645. The PID_I calculation controller 645 accounts for the past values of the TCS(T) – Liquid(T) error value and integrates the past error values over time to produce the integral (I) term. For example, if there is a residual TCS(T) – Liquid(T) error value after the application of proportional (P) control, the integral (I) term seeks to eliminate the residual error by adding a control effect due to the historic cumulative value of the TCS(T) – Liquid(T) error value. When the error is eliminated, the integral term ceases to grow. This results in the proportional effect diminishing as the error decreases, but this is compensated for by the growing integral effect. Next, the MCU 465 in 650 sums the PID_P, PID_D, and PID_I components to produce an adjustment value for the power setting of the TCS 401.

If No in 625, the MCU 465 in 635 back calculates the theoretical TCS 401 power, based on the thermal mass, the specific heat of the liquid, the temperature difference (ΔT) between samples, and the time difference (Δt) between samples. In 640, the MCU 465 uses the theoretical TCS power value from 635 and a previous TCS 401 power level to calculate a heat loss value. This heat loss value is utilized in calculation 655 to modify or curtail the PID_I accumulation value, which is an input to the PID_I calculation controller 645. Then, the MCU 465 in 650 sums the PID_P, PID_D, and PID_I components to produce an adjustment value for the power setting of the TCS 401.

In embodiments that utilize a glycol-based thermal liquid rather than pure water, the PID control algorithm implemented by the MCU 465 operates with modified coefficients and response characteristics to account for the different thermophysical properties of the glycol-water mixture. The thermal conductivity of a glycol-water mixture is lower than that of pure water, and the viscosity is much higher, meaning that the system response may be much slower, even though the bulk fluid experiences thermal change quicker. This change in performance affects the thermal response characteristics of the system and requires corresponding adjustments to the PID coefficients.

In water-based embodiments, the proportional gain coefficient (K) may be set to a first value that provides appropriate responsiveness given the thermal mass and heat capacity of the water. In glycol-based embodiments, the proportional gain coefficient may be reduced to a second value that is lower than the first value, such as a reduction of approximately 15% to 25%, to account for the lag in temperature change of the glycol-based thermal liquid. Without this reduction, the same power input would produce a larger temperature change in the glycol-based system, potentially causing overshoot or oscillation around the target setpoint.

The integral component of the PID algorithm is particularly affected by the transition from water to a glycol-based thermal liquid. In water-based systems, the integral component accumulates error over time to eliminate steady-state offset between the target temperature and the actual temperature. The rate of accumulation and the maximum allowable magnitude of the integral term are calibrated based on the thermal response characteristics of water. In glycol-based embodiments, the integral component must be dampened relative to water-based embodiments because the slower thermal response of the glycol-based mixture means that the integral component will be accumulating over a much larger time interval. If the integral component were allowed to accumulate at the same rate as in water-based systems, the resulting control output would be excessive for the glycol-based thermal liquid, causing overshoot and subsequent oscillation as the system attempts to correct for the overshoot.

The damping of the integral component in glycol-based embodiments may be implemented by reducing the integral gain coefficient, by reducing the maximum allowable magnitude of the accumulated integral term, or by both approaches in combination. For example, the integral gain coefficient in a glycol-based embodiment may be reduced by approximately 20% to 30% compared to a water-based embodiment, and the maximum allowable magnitude of the accumulated integral term may be reduced by a similar proportion. These adjustments ensure that the integral component provides sufficient correction to eliminate steady-state error without causing the excessive control output that would result from applying water-calibrated integral parameters to a glycol-based system.

Lastly, the derivative coefficient in glycol-based embodiments may be increased to counteract oscillation. Because the glycol-based thermal liquid responds more slowly to heating and cooling inputs, there is an increased chance for overshoot and oscillation. The derivative component of the PID algorithm, which responds to the rate of change of the error value, may therefore be increased in glycol-based embodiments to provide greater damping effect and prevent oscillatory behavior that could otherwise result from the slower system response.

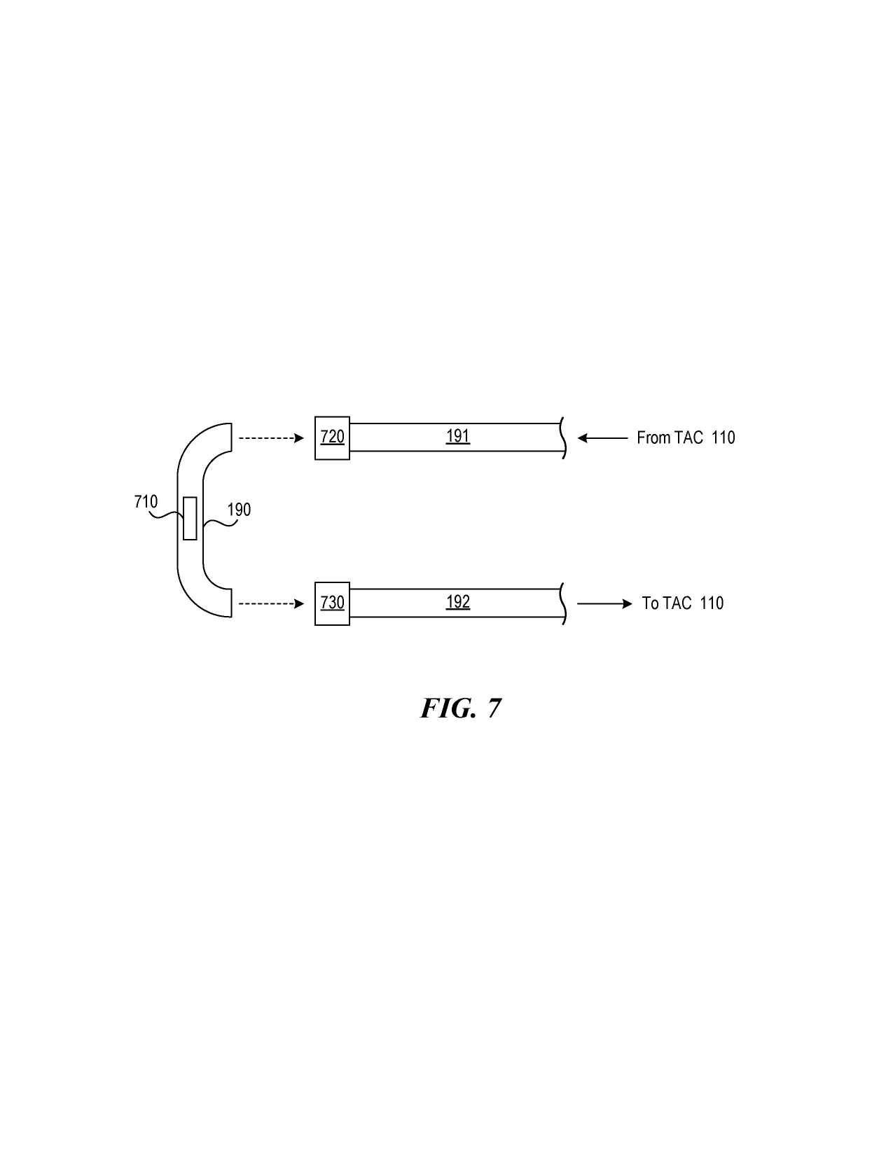

FIG. 7 illustrates the components of a loop back connector 190 used to decontaminate the TAC 110 according to one embodiment of the disclosure. An operator couples a first end of the loop back connector 190 to a connector 720 at a distal end of the input conduit 191 and couples a second end of the loop back connector 190 to a connector 730 at a distal end of the output conduit 192. In some embodiments, the connector 720 and the connector 730 are also used to secure the thermal accessory 160 to the TAC 110. In embodiments with multiple thermal accessories 160, the connectors 720 and 730 are multiple connectors 720 and 730 that each secure a different one of multiple loop back connectors 190 to the TAC 110 such that all input and output ports of the TAC 110 are connected to a loop back connector. The loop back connector 190 provides a liquid path directly from the input conduit 191 to the output conduit 192 when the thermal blanket 160 is disconnected for cleaning operations.

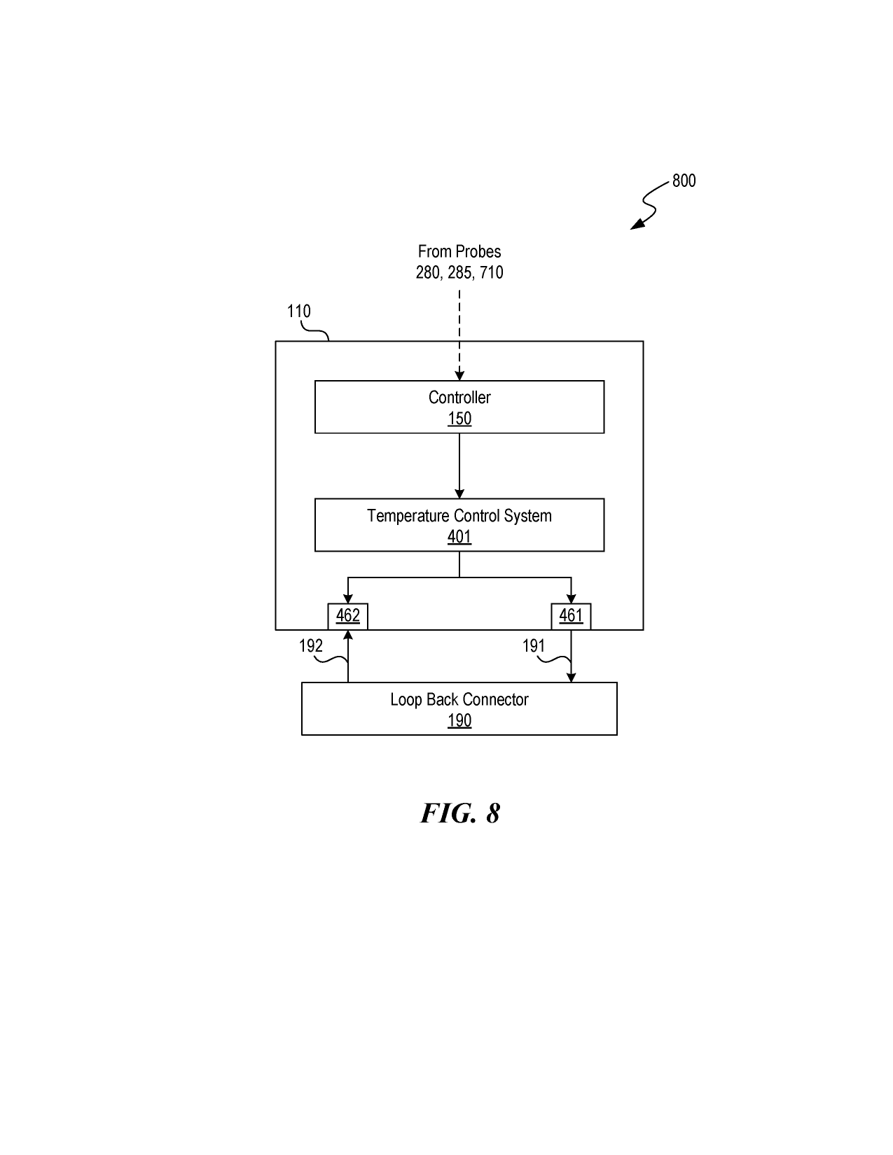

In some embodiments, the loop back connector 190 includes a loop back probe 710 that connects to a patient temperature probe port on the TAC 110. The loop back probe 710 serves a dual purpose in the disinfection procedure (e.g., as described below with respect to FIG. 8). First, the MCU 465 continuously compares the temperature readings from the loop back probe 710 to the temperature readings from the internal fluid sensors 470 and 475. If the loop back probe 710 measures approximately the same temperature as the internal fluid sensors, the MCU 465 can confirm that the TAC 110 is circulating thermal liquid through the loop back connector 190 rather than through a thermal accessory connected to a patient. This comparison is expected to provide a safeguard against inadvertently exposing a patient to thermal liquid at temperatures exceeding safe limits (e.g., temperatures above approximately 42°C).

Second, the loop back probe 710 can verify that the thermal liquid temperature throughout the entire fluid path exceeds 70°C for a sufficient duration to achieve effective microbial decontamination. By positioning the loop back probe 710 at the most distant point from the TAC 110 in the fluid path, the MCU 465 can confirm that even the coolest portion of the circulating thermal liquid has reached the required disinfection temperature. This is expected to ensure that all internal plumbing components of the TAC 110 are exposed to the elevated temperature necessary to kill microbes.

In some embodiments, to prevent an operator from inadvertently using incorrect temperature probes during the disinfection procedure, the loop back probe 710 comprises a resistance temperature detector (RTD) that operates on a temperature-resistance curve distinct from the patient temperature probes described above (e.g., the patient temperature probes 280 and 285). The MCU 465 measures the resistance of the connected probes and analyzes the temperature-resistance characteristics to determine whether a loop back probe 710 or patient temperature probe are connected. The MCU 465 can permit the 70°C disinfection procedure to proceed only when the MCU 465 detects that a loop back probe 710 is connected to the patient temperature probe ports. If the MCU 465 detects that patient temperature probes 280 and 285 are connected instead of a loop back probe 710, the MCU 465 can prevent the disinfection procedure from initiating, thereby reducing the risk of exposing a patient to dangerously high thermal liquid temperatures.

In some embodiments, the loop back probe 710 may communicate with the TAC 110 by a wireline (not shown) in the loop back connector 190. In other embodiments, the loop back probe 710 may communicate with the TAC 110 wirelessly by, for example, a Bluetooth transceiver (not shown) in the housing of the loop back probe 710 itself.

Accordingly, the loop back connector 190 is expected to ensure that the TAC 110 is not connected to a thermal blanket 160 and to reduce the risk of misuse of the device. Once the loop back connector 190 is connected, the operator can press a button on the UI 210, which starts the cleaning and disinfection procedure. The operator is then free to leave the TAC 110 unattended and return when the procedure is finished. The MCU 465 can automatically shut off the cleaning and disinfection procedure after a set period.

FIG. 8 is a flow diagram 800 showing the operation of the decontamination procedure of the thermal accessory controller according to one embodiment of the disclosure. The components used in the decontamination procedure comprise the TAC 110, which includes a controller 150, the TCS 401, an input port 462, an output port 461, a loop back connector 190, an input liquid conduit 191 to the loop back connector, and an output liquid conduit 192 from the loop back connector. The circulating liquid used for normal operation, the decontamination procedure, or both normal operation and the decontamination procedure may include a residual disinfectant and/or an antimicrobial liquid.

The input to the controller 150 within the TAC 110 is temperature information from one or more probes (e.g., temperature probes 280 and 285 or a loop back probe 710). The controller 150 measures the resistance of the probe(s) connected to the TAC 110 and detects whether temperature probes 280 and 285 or a loop back probe 710 is connected through the use of temperature-resistance curves. The decontamination procedure using 70°C liquid will only occur if the controller 150 detects the use of a loop back probe 710.

To verify that the loop back connector 190 is attached to the TAC 110, the TCS 401 ensures that the temperature of the liquid within the device matches the temperature of the loop back connector 190. The liquid flows from the TCS 401, through the output port 461 of the TAC 110, the input liquid conduit 191, the loop back connector 190, the output liquid conduit 192, and back into the TAC 110 through the input port 462. The liquid path touches all internal plumbing in the TAC 110 to ensure each component is decontaminated.

Unique aspects of the disclosed system and method include the use of a glycol liquid to reduce antimicrobial load and a cleaning mode that uses 70°C liquid to kill microbes in the TAC 110. More specifically, the glycol liquid creates an unsuitable environment for microbial growth through mechanisms that include, but are not limited to, decreasing surface tension, chemical structure, osmotic effect, chemical additives, and alkalinity. The glycol liquid also increases the boiling point to achieve higher temperatures and without loss (via evaporation) of usable solution volume in liquid state flowing through the system. The glycol liquid decreases the freezing point to achieve colder temperatures and to prevent solidification of solution that may cause clogging at lower temperatures. Furthermore, the coloration of the glycol liquid improves safety by clearly distinguishing the appropriate liquid to be used and by making it easier to detect leaks, inhibited flow, or bubbles.

Although the present disclosure has been described with an exemplary embodiment, various changes and modifications may be suggested to one skilled in the art. It is intended that the present disclosure encompass such changes and modifications as fall within the scope of the appended claims.

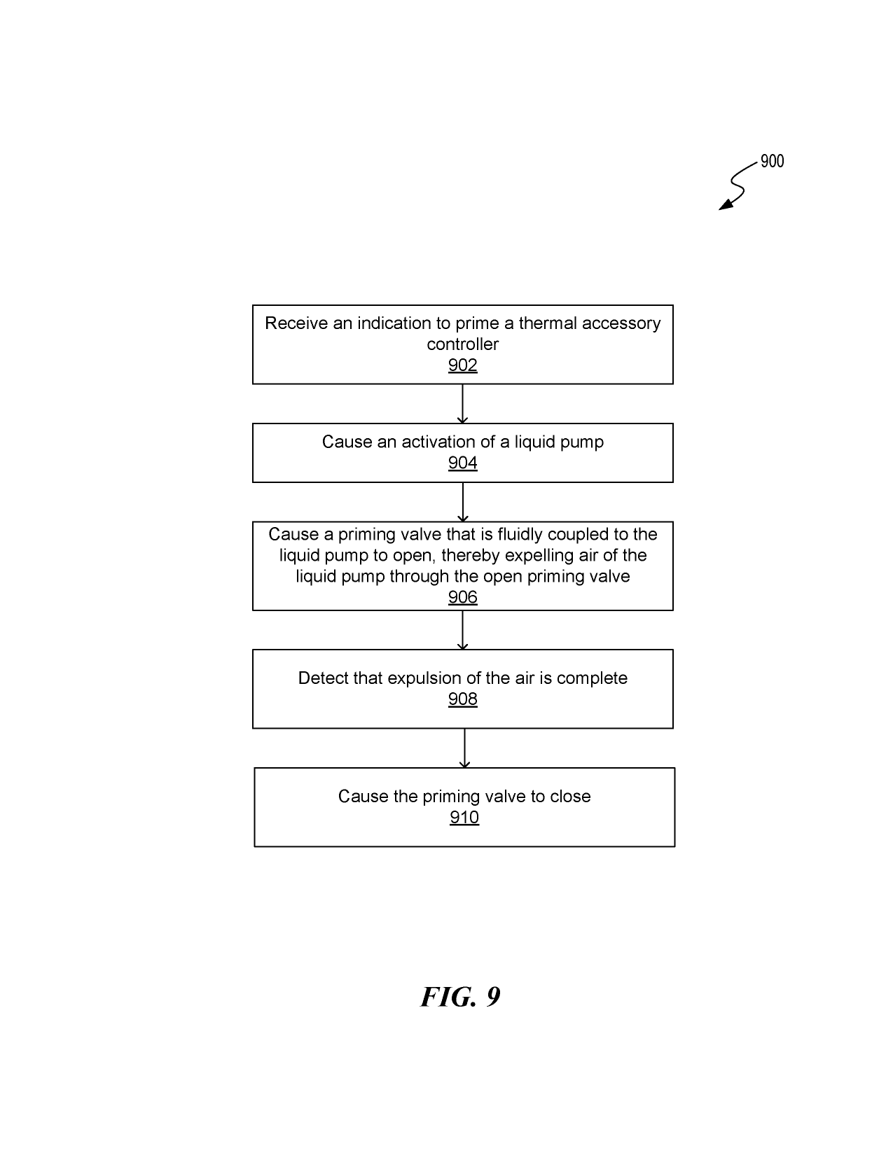

FIG. 9 is a flowchart of a process 900 for priming a patient temperature management system (e.g., the patient temperature management system 100 of FIG. 1). At 902, a control unit (e.g., the MCU 465 of FIGS. 4A-B) receives an indication to prime the thermal accessory controller (e.g., the TAC 110 of FIG. 1). The indication may be received from an operator input through a user interface (e.g., the UI 210 of FIG. 2) or may be automatically triggered during an initial setup procedure. At 904, the control unit causes an activation of a liquid pump (e.g., the liquid pump 130 of FIG. 1). At 906, the control unit causes a priming valve (e.g., the priming valve 450 of FIGS. 4A-B) that is fluidly coupled to the liquid pump to open, expelling air within the liquid pump through the open priming valve. The priming valve provides a pathway for the air to escape from the liquid pump and the surrounding fluid pathways, which is expected to enable the thermal liquid to fully flood the pump chamber. At 908, the control unit detects that expulsion of the air is complete. The detection may be based on a predetermined time period, a change in pressure within the fluid path, a change in flow rate detected by a flow sensor, or visual confirmation by an operator through the user interface. At 910, the control unit causes the priming valve to close. Once the priming valve is closed, the liquid pump 130 is primed and ready for normal operation, with the liquid pump capable of circulating thermal liquid through the fluid path with a reduced risk of cavitation or flow interruptions caused by residual air pockets.

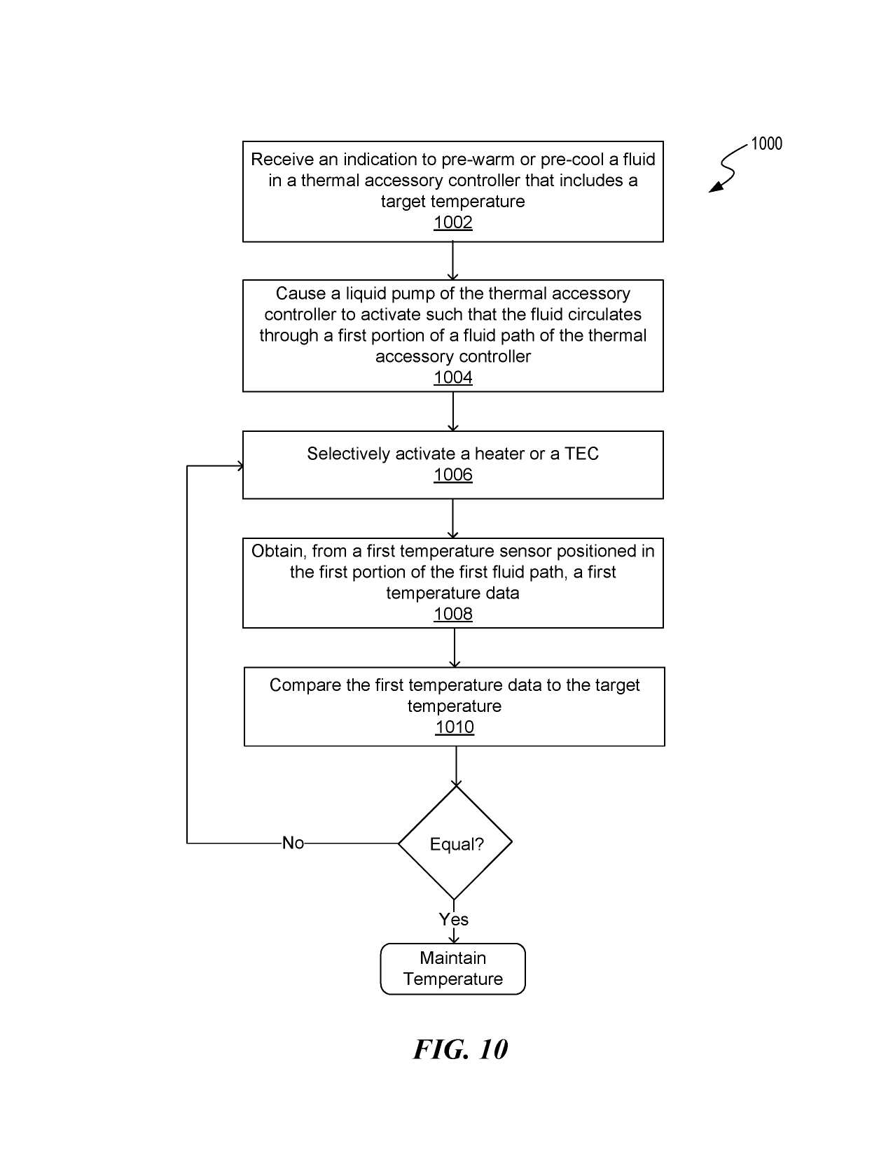

FIG. 10 is a flowchart of a process 1000 for pre-warming or pre-cooling fluid in a patient temperature management system (e.g., the patient temperature management system 100 of FIG. 1). At 1002, a control unit (e.g., the MCU 465 of FIGS. 4A-B) receives an indication to pre-warm or pre-cool a fluid in a thermal accessory controller (e.g., the TAC 110 of FIG. 1) that includes a target temperature. In some embodiments, the indication may be received from an operator input through a user interface (e.g., the UI 210 of FIG. 2) specifying the desired target temperature for the thermal liquid. In other embodiments, the indication is received from an operator input through a user interface that places the system in the normal RUN mode, which causes the fluid in the thermal accessory controller to pre-warm or pre-cool when no thermal accessory is attached. At 1004, the control unit causes a liquid pump (e.g., the liquid pump 130 of FIG. 1) of the thermal accessory controller to activate such that the fluid circulates through a first portion of a fluid path of the thermal accessory controller. In some embodiments, the first portion of the fluid path is similar to the first portion of the fluid path described above with respect to FIG. 4A. In such embodiments, when no thermal accessory is attached, the fluid circulates through the check valve 460 and returns to the liquid reservoir 140.

At 1006, the control unit selectively activates a heater (e.g., the heater 120 of FIG. 1) or a TEC (e.g., the TEC 425 and TEC 426 of FIGS. 4A-B) depending on whether the target temperature is above or below the current fluid temperature. At 1008, the control unit obtains, from a first temperature sensor (e.g., the sensor 470 of FIGS. 4A-B) positioned in the first portion of the fluid path, first temperature data representing the current temperature of the circulating fluid. At 1010, the control unit compares the first temperature data to the target temperature. If the first temperature data equals the target temperature (or is within an acceptable tolerance of the target temperature), the thermal accessory controller maintains this temperature and is ready for connection to a thermal accessory at the desired temperature. If the first temperature data does not equal the target temperature, the process returns to step 1006 to continue selectively activating the heater or the TEC until the target temperature is achieved.

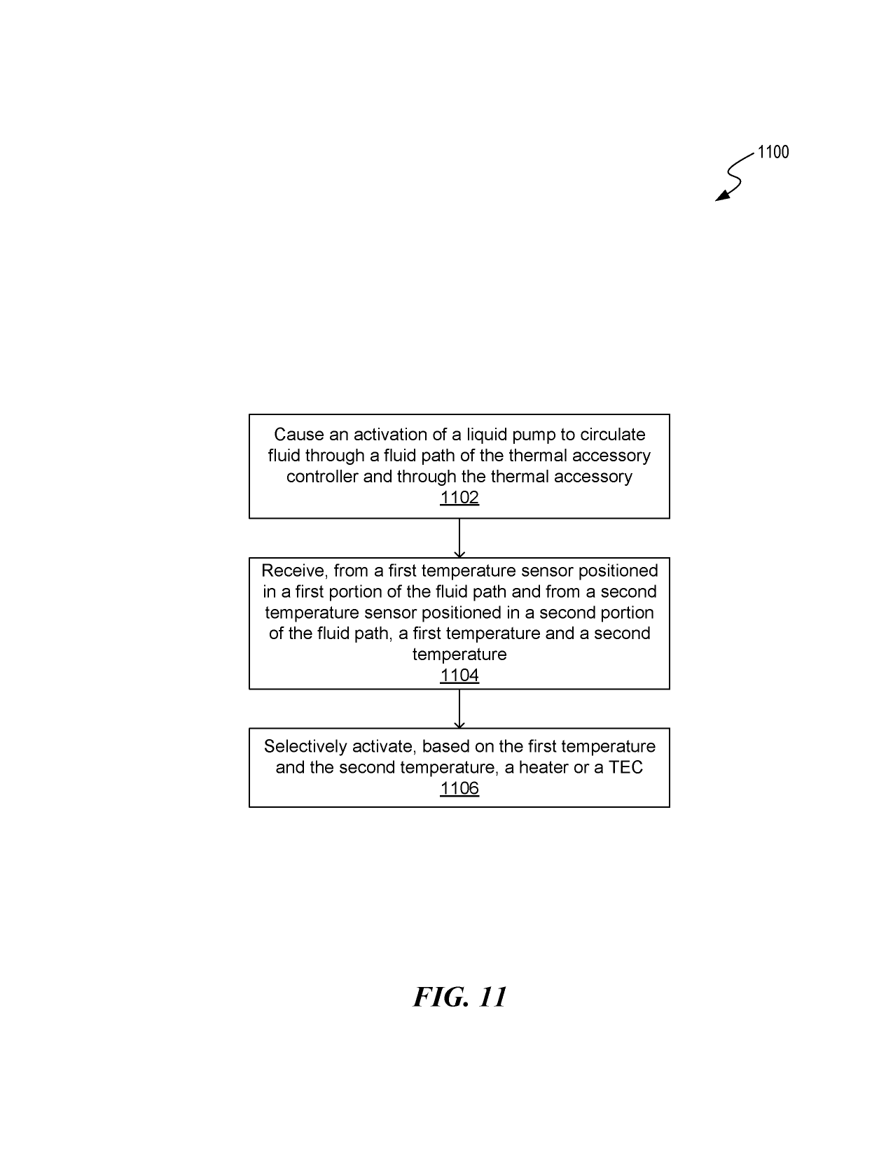

FIG. 11 is a flowchart of a process 1100 for monitoring a temperature of a fluid in a patient temperature management system (e.g., the patient temperature management system 100 of FIG. 1) when a thermal accessory is attached. At 1102, the control unit causes an activation of a liquid pump (e.g., the liquid pump 130 of FIG. 1) to circulate fluid through a fluid path of the thermal accessory controller and through the thermal accessory.

At 1104, the control unit receives, from a first temperature sensor (e.g., the sensor 470 of FIGS. 4A-B) positioned in a first portion of the fluid path and from a second temperature sensor (e.g., the sensor 475 of FIGS. 4A-B) positioned in a second portion of the fluid path, a first temperature and a second temperature. In some embodiments, the first portion of the fluid path includes the pathway through the check valve 460, while the second portion of the fluid path includes the pathway leading to the thermal accessory 160 as shown in FIGS. 4A-B. The first temperature and the second temperature provide the control unit with information regarding the thermal state of the circulating fluid at different points in the fluid path.

At 1106, the control unit selectively activates, based on the first temperature and the second temperature, a heater (e.g., the heater 120 of FIG. 1) or a TEC (e.g., the TEC 425 and TEC 426 of FIGS. 4A-B). The control unit may compare the first temperature and the second temperature to a target temperature setpoint and determine whether heating or cooling is required to achieve the target temperature. In some embodiments, the control unit averages the first and second temperature, takes a weighted average of the first and second temperature, or selects one of the first and second temperature to compare with the target temperature. In heating mode, the control unit activates the heater to add thermal energy to the circulating fluid. In cooling mode, the control unit activates the TEC to remove thermal energy from the circulating fluid.

### Computer System

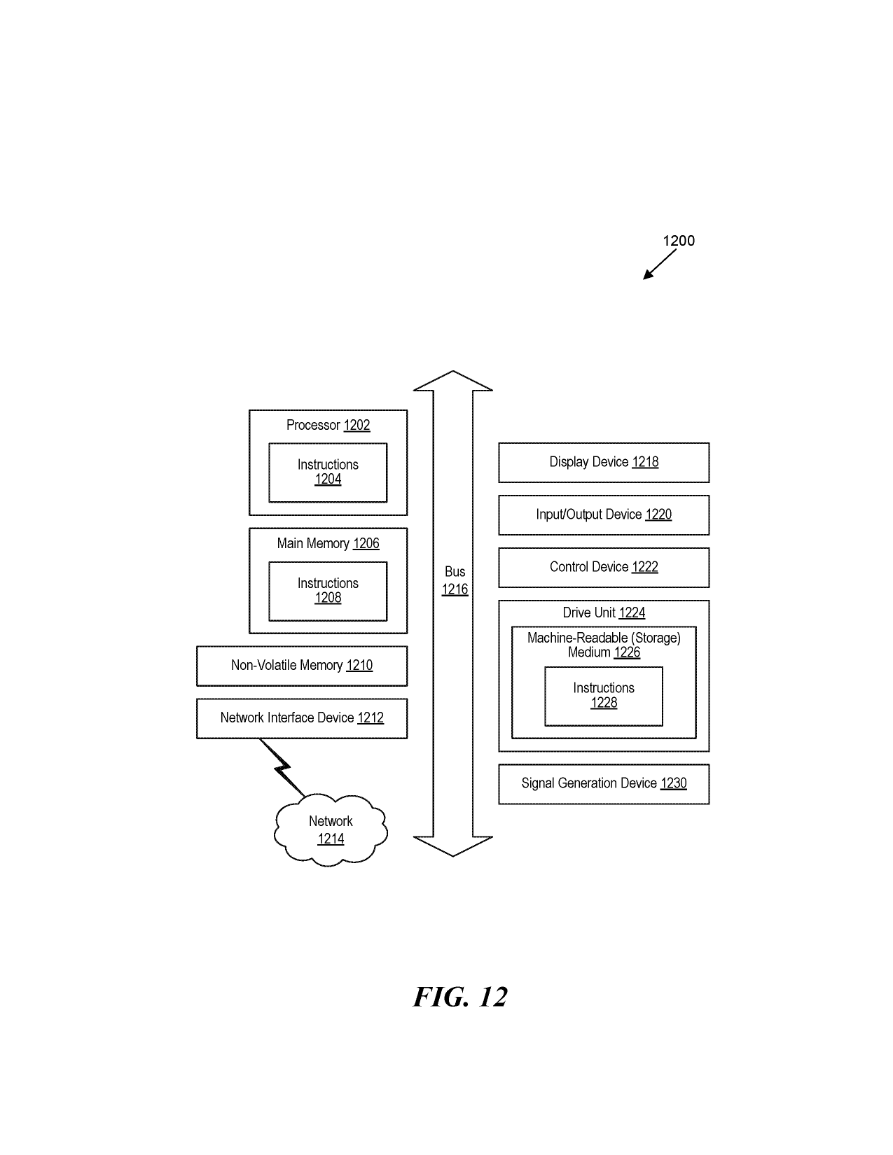

FIG. 12 is a block diagram that illustrates an example of a computer system 1200 in which at least some operations described herein can be implemented. As shown, the computer system 1200 can include: one or more processors 1202, main memory 1206, non-volatile memory 1210, a network interface device 1212, a video display device 1218, an input/output device 1220, a control device 1222 (e.g., keyboard and pointing device), a drive unit 1224 that includes a machine-readable (storage) medium 1226, and a signal generation device 1230 that are communicatively connected to a bus 1216. The bus 1216 represents one or more physical buses and/or point-to-point connections that are connected by appropriate bridges, adapters, or controllers. Various common components (e.g., cache memory) are omitted from FIG. 12 for brevity. Instead, the computer system 1200 is intended to illustrate a hardware device on which components illustrated or described relative to the examples of the figures and any other components described in this specification can be implemented.

The computer system 1200 can take any suitable physical form. For example, the computing system 1200 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computing system 1200. In some implementations, the computer system 1200 can be an embedded computer system, a system-on-chip (SOC), a single-board computer system (SBC), or a distributed system such as a mesh of computer systems, or it can include one or more cloud components in one or more networks. Where appropriate, one or more computer systems 1200 can perform operations in real time, in near real time, or in batch mode.

The network interface device 1212 enables the computing system 1200 to mediate data in a network 1214 with an entity that is external to the computing system 1200 through any communication protocol supported by the computing system 1200 and the external entity. Examples of the network interface device 1212 include a network adapter card, a wireless network interface card, a router, an access point, a wireless router, a switch, a multilayer switch, a protocol converter, a gateway, a bridge, a bridge router, a hub, a digital media receiver, and/or a repeater, as well as all wireless elements noted herein.

The memory (e.g., main memory 1206, non-volatile memory 1210, machine-readable medium 1226) can be local, remote, or distributed. Although shown as a single medium, the machine-readable medium 1226 can include multiple media (e.g., a centralized/distributed database and/or associated caches and servers) that store one or more sets of instructions 1228. The machine-readable medium 1226 can include any medium that is capable of storing, encoding, or carrying a set of instructions for execution by the computing system 1200. The machine-readable medium 1226 can be non-transitory or comprise a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory 1210, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically comprise one or more instructions (e.g., instructions 1204, 1208, 1228) set at various times in various memory and storage devices in computing device(s). When read and executed by the processor 1202, the instruction(s) cause the computing system 1200 to perform operations to execute elements involving the various aspects of the disclosure.

### Remarks

The terms “example,” “embodiment,” and “implementation” are used interchangeably. For example, references to “one example” or “an example” in the disclosure can be, but not necessarily are, references to the same implementation; and such references mean at least one of the implementations. The appearances of the phrase “in one example” are not necessarily all referring to the same example, nor are separate or alternative examples mutually exclusive of other examples. A feature, structure, or characteristic described in connection with an example can be included in another example of the disclosure. Moreover, various features are described that can be exhibited by some examples and not by others. Similarly, various requirements are described that can be requirements for some examples but not for other examples.

The terminology used herein should be interpreted in its broadest reasonable manner, even though it is being used in conjunction with certain specific examples of the invention. The terms used in the disclosure generally have their ordinary meanings in the relevant technical art, within the context of the disclosure, and in the specific context where each term is used. A recital of alternative language or synonyms does not exclude the use of other synonyms. Special significance should not be placed upon whether or not a term is elaborated or discussed herein. The use of highlighting has no influence on the scope and meaning of a term. Further, it will be appreciated that the same thing can be said in more than one way.

Unless the context clearly requires otherwise, throughout the description and the claims, the words “comprise,” “comprising,” and the like are to be construed in an inclusive sense, as opposed to an exclusive or exhaustive sense—that is to say, in the sense of “including, but not limited to.” As used herein, the terms “connected,” “coupled,” and any variants thereof mean any connection or coupling, either direct or indirect, between two or more elements; the coupling or connection between the elements can be physical, logical, or a combination thereof. Additionally, the words “herein,” “above,” “below,” and words of similar import can refer to this application as a whole and not to any particular portions of this application. Where context permits, words in the above Detailed Description using the singular or plural number may also include the plural or singular number, respectively. The word “or” in reference to a list of two or more items covers all of the following interpretations of the word: any of the items in the list, all of the items in the list, and any combination of the items in the list. The term “module” refers broadly to software components, firmware components, and/or hardware components. Further, the term “approximately” generally refers to a concentration or amount of a substance within a 10 percent range above or below a stated percentage.

While specific examples of technology are described above for illustrative purposes, various equivalent modifications are possible within the scope of the invention, as those skilled in the relevant art will recognize. For example, while processes or blocks are presented in a given order, alternative implementations can perform routines having steps, or employ systems having blocks, in a different order, and some processes or blocks may be deleted, moved, added, subdivided, combined, and/or modified to provide alternative or sub-combinations. Each of these processes or blocks can be implemented in a variety of different ways. Also, while processes or blocks are at times shown as being performed in series, these processes or blocks can instead be performed or implemented in parallel, or can be performed at different times. Further, any specific numbers noted herein are only examples such that alternative implementations can employ differing values or ranges.

Details of the disclosed implementations can vary considerably in specific implementations while still being encompassed by the disclosed teachings. As noted above, particular terminology used when describing features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed herein, unless the above Detailed Description explicitly defines such terms. Accordingly, the actual scope of the invention encompasses not only the disclosed examples but also all equivalent ways of practicing or implementing the invention under the claims. Some alternative implementations can include additional elements to those implementations described above or include fewer elements.

We claim:

1. A system for controlling a temperature of a thermal accessory, the system comprising:
a fluid path between an input port of the system and an output port of the system, the fluid path including:
a liquid pump that is configured to move thermal liquid along the fluid path, the thermal liquid comprising a mixture of (i) water, (ii) propylene glycol, and (iii) a corrosion inhibitor;
a liquid reservoir that includes a store of the thermal liquid;
a heating component;
a cooling component;
a first portion of the fluid path, the first portion of the fluid path including:
a valve, the valve configured to (i) restrict the thermal liquid from entering the liquid reservoir from the first portion of the fluid path when the thermal accessory is coupled to the system and (ii) permit the thermal liquid to enter the liquid reservoir from the first portion of the fluid path when the thermal accessory is decoupled from the system; and
a second portion of the fluid path, the second portion of the fluid path comprising:
the output port, the output port configured to (i) permit the thermal liquid to exit the system from the output port when the thermal accessory is coupled to the system and (ii) restrict the thermal liquid from exiting the system from the output port when the thermal accessory is decoupled from the system; and
a control unit configured to manage the temperature of the thermal accessory by either (i) heating the thermal liquid through controlled activation of the heating component or (ii) cooling the thermal liquid through controlled activation of the cooling component.

2. The system of claim 1,
wherein the thermal liquid comprises propylene glycol in an amount of 20 to 100 percent by volume, and
wherein the thermal liquid comprises water in an amount of 0 to 80 percent by volume.

3. The system of claim 1, wherein the liquid pump further comprises:
an impeller; and
a tachometer that is configured to –
measure a first rotation speed of the impeller, and
transmit the first rotation speed to the control unit,
wherein, in response to the first rotation speed falling below a first threshold, the control unit causes the impeller to rotate at a second rotation speed that is higher than the first rotation speed, thereby increasing a flow rate of the thermal liquid through the fluid path.

4. The system of claim 1, wherein the second portion of the fluid path further comprises:
a flow sensor that is configured to –
measure a first flow rate of the thermal liquid through the fluid path, and
transmit the first flow rate to the control unit,
wherein, in response to the first flow rate falling below a second threshold, the control unit causes the liquid pump to move the thermal liquid along the fluid path at a second flow rate that is higher than the first flow rate.

5. The system of claim 1, wherein the mixture further comprises a pH-activated indicator that is configured to provide a visual indication of a pH of the thermal liquid.

6. The system of claim 1, wherein the mixture further comprises:
a bactericidal additive;
a thermal-conductivity additive;
a heat-capacity additive;
a pH indicator;
a stabilizer; or
an antioxidant.

7. A system for controlling temperature of a bodily fluid through thermal transfer between the bodily fluid and a solution that is fully contained in the system and that includes:
(i) water in an amount of 30 to 70 percent by volume, and(ii) a monomeric glycol in an amount of 30 to 70 percent by volume.

8. The system of claim 7, wherein the solution further includes:
a corrosion inhibitor in an amount that is no more than 5% by volume.

9. The system of claim 7, the system further comprising:
a fluid path between an input port of the system and an output port of the system, the fluid path including:
a liquid pump that is configured to move the solution along the fluid path;
a liquid reservoir that includes a store of the solution;
a heating component;
a cooling component; and
a control unit configured to manage the temperature of the solution by either (i) heating the solution through controlled activation of the heating component or (ii) cooling the solution through controlled activation of the cooling component.

10. The system of claim 9, wherein the liquid pump further comprises:
an impeller; and
a tachometer that is configured to –
measure a first rotation speed of the impeller, and
transmit the first rotation speed to the control unit,
wherein, in response to the first rotation speed falling below a first threshold, the control unit causes the impeller to rotate at a second rotation speed that is higher than the first rotation speed, thereby increasing a flow rate of the solution through the fluid path.

11. The system of claim 9, the system further comprising:
a flow sensor that is configured to –
measure a first flow rate of the solution through the fluid path, and
transmit the first flow rate to the control unit,
wherein, in response to the first flow rate falling below a second threshold, the control unit causes the liquid pump to move the solution along the fluid path at a second flow rate that is higher than the first flow rate.

12. The system of claim 7, wherein the monomeric glycol comprises propylene glycol.

13. The system of claim 7, wherein the solution further includes a pH-activated indicator that is configured to provide a visual indication of a pH of the solution.

14. The system of claim 7, wherein the solution further includes:
a bactericidal additive;
a thermal-conductivity additive;
a heat-capacity additive;
a pH indicator;
a stabilizer; or
an antioxidant.

15. A system for controlling a temperature of a thermal accessory, the system comprising:
a fluid path between an input port of the system and an output port of the system, the fluid path including:
a pump that is configured to move a glycol-based fluid that comprises at least 30 percent of a monomeric glycol by volume along the fluid path;
a reservoir that includes a store of the glycol-based fluid;
a heating component;
a cooling component;
a first portion of the fluid path, the first portion of the fluid path including:
a valve that is configured to (i) restrict the glycol-based fluid from entering the reservoir from the first portion of the fluid path when the thermal accessory is coupled to the system and (ii) permit the glycol-based fluid to enter the reservoir from the first portion of the fluid path when the thermal accessory is decoupled from the system; and
a second portion of the fluid path, the second portion of the fluid path comprising:
the output port, the output port configured to (i) permit the glycol-based fluid to exit the system from the output port when the thermal accessory is coupled to the system and (ii) restrict the glycol-based fluid from exiting the system from the output port when the thermal accessory is decoupled from the system; and
a control unit configured to manage the temperature of the thermal accessory by controlled activation of at least one of the heating component and the cooling component.

16. The system of claim 15, wherein the glycol-based fluid comprises at least 50 percent of a monomeric glycol by volume.

17. The system of claim 15, wherein the glycol-based fluid comprises at least 70 percent of a monomeric glycol by volume.

18. The system of claim 15, wherein the monomeric glycol is propylene glycol, and wherein the glycol-based fluid further comprises a corrosion inhibitor in an amount no more than 5 percent by volume.

19. The system of claim 15, wherein the glycol-based fluid further comprises a pH-activated indicator that is configured to provide a visual indication of a pH of the glycol-based fluid.

20. The system of claim 15, wherein the glycol-based fluid further comprises:
a bactericidal additive;
a thermal-conductivity additive;
a heat-capacity additive;
a pH indicator;
a stabilizer; or
an antioxidant.
